(19) United States
(12) Patent Application Publication
(10) Pub. No.: US 2012/0058091 A1
(43) Pub. Date: Mar. 8, 2012

(54) DIAGNOSTIC AND THERAPEUTIC TREATMENTS RELATED TO MITOCHONDRIAL DISORDERS

(75) Inventors: Martha Karen Newell Rogers, Holland, TX (US); Elizabeth Villalobos-Menuey, Monument, CO (US); Robert Melamede, (US); Robert E. Camley, Colorado Springs, CO (US)

(73) Assignee: The Regents of the University of Colorado, Denver, CO (US)

(21) Appl. No.: 13/074,922

(22) Filed: Mar. 29, 2011

Related U.S. Application Data
(62) Division of application No. 10/556,653, filed on Sep. 7, 2007, now abandoned.
(60) Provisional application No. 60/470,046, filed on May 13, 2003.

(51) Int. Cl. A61K 48/00; A61P 25/00; C12N 5/16

(52) U.S. Cl. 424/93.21; 435/346

(57) ABSTRACT

The invention is based in part on the discovery that chromosomal disorders such as Down Syndrome can be diagnosed by assessing maternal mitochondrial status. Thus the invention relates to diagnostic and therapeutic methods and related products for chromosomal disorders such as Downs Syndrome, for example, for identifying a risk of fetal Downs Syndrome and methods of mitigating that risk. The methods also are useful for other therapies where it is desirable to manipulate mitochondria such as tissue generation.

### FIELD OF THE INVENTION

The invention relates to diagnostic and therapeutic methods and related products for chromosomal disorders such as Down Syndrome. The methods and products are useful, for example, for identifying a risk of Down Syndrome and methods of mitigating that risk. The methods also are useful for other therapies where it is desirable to manipulate mitochondria such as tissue generation.

### BACKGROUND OF THE INVENTION

Down Syndrome is the most common aneuploidy and serious cognitive disorder at birth (Jacobs, P., et al 1959. The somatic chromosomes in mongolism. Lance 1:710-711. Arbuzova, S., et al 2002. Mitochondrial dysfunction and Down's syndrome. BioEssays 24: 681. Lejeune, J., et al. 1959. Etudes des chromosomes somatiques de neau enfants mongliens. CR Acad Sci (Paris) 248: 1721). Neither the pathogenesis nor the etiology of Down Syndrome is understood. Children with Down Syndrome suffer many diseases including cardiovascular diseases, increased susceptibility to infections, leukemia, endocrine alterations, immune defects, nutritional disturbance, increased and early susceptibility to Alzheimer's Disease (Loft, I. T., and Head, E. 2001. Down Syndrome and Alzheimer's disease: a link between development and aging. Ment. Retard. Dev. Disabil. Res. Rev: 7: 172), and cognitive disabilities. Children with Down Syndrome have to cope with a significant pro-oxidant environment. Oxidative stress can contribute to atherosclerosis, early aging, immunological deficiencies, and neurologic disorders in Down Syndrome patients.

The mechanism by which trisomy of chromosome 21 produces multiple pathologies is not known. The chromosome has now been completely sequenced and it includes a number of important genes in energy metabolism, including genes involved in regulating oxidative processes (Hattori, M. et. al. 2000. The chromosome 21 mapping and sequencing consortium. The DNA sequence of human chromosome 21. Nature 405: 311). These include the genes for Cu, Zn superoxide dismutase (SOD-1) (Arbuzova, S., et al 2002. Mitochondrial dysfunction and Down's syndrome. BioEssays 24: 681.) (Schuchmann, S., and Heinemann, U. 2000. Increased mitochondrial superoxide generation in neurons from Down's Syndrome. Free Radio Biol Med 28: 235) and the amyloid precursor protein (Busciglio, J., et al. 2002. Altered metabolism of the amyloid beta precursor protein is associated with mitochondrial dysfunction in Down's Syndrome. Neuron 33: 677), among many others. However, there is no direct evidence that any locus alone is responsible for any of physiological features of the syndrome (Schon, E. A., et al 2000. Chromosomal non-disjunction in human oocytes: is there a mitochondrial connection? Human Reprod. 15: 160).

### SUMMARY OF THE INVENTION

In some aspects the invention is a diagnostic method. The method is performed by assessing mitochondrial status in a maternal sample, wherein a mitochondrial deletion associated with altered metabolic activity is predictive of a pre-disposition to a chromosomal abnormality associated with Down Syndrome in a fetus. In other aspects the diagnostic method is performed by assessing mitochondrial status in a maternal sample, wherein a level of mitochondrial membrane potential that is less than a normal baseline value of mitochondrial membrane potential is predictive of a pre-disposition to a chromosomal abnormality associated with Down Syndrome in a fetus.

In some embodiments the maternal sample is peripheral blood. In other embodiments the maternal sample is isolated from a subject prior to assessment of mitochondrial status. In yet other embodiments the diagnostic method is performed on a subject prior to conception. Alternatively the diagnostic method is performed on a subject after conception.

The method may also involve the performance of amniocentesis after assessing the mitochondrial status.

In some embodiments the mitochondrial status is determined by a quantitative measure of electron potential, for instance, using mitotracker red. In other embodiments the mitochondrial status is determined by a detection of cell surface molecule expression, such as MHC class I, MHC class II, fas, B71, B72, CD40, fas ligand, or cell surface UCP.

The mitochondrial deletion may be a deletion in complex I genes of mitochondrial DNA.

A method of modifying an oocyte or embryonic cell is provided according to other aspects. The method involves microinjecting a heterologous mitochondria into an oocyte or embryonic cell wherein the heterologous mitochondria is capable of achieving at least normal levels of mitochondrial membrane potential in the oocyte or embryonic cell. In some embodiments the heterologous mitochondria is microinjected in vitro and the oocyte or embryonic cell is then implanted into a subject. In other embodiments the oocyte is derived from a subject determined to have a pre-disposition to a chromosomal abnormality associated with Down Syndrome in a fetus.

A modified stem cell, comprising a stem cell having a heterologous mitochondria. The heterologous mitochondria may have a level of mitochondrial membrane potential that is within a normal range relative to a healthy stem cell.

In other aspects the invention is a method for promoting tissue generation, comprising subjecting the modified stem cell of the invention to growth promoting conditions. The modified stem cell may be implanted into a subject. In some embodiments the modified stem cell is autologous to the subject. In other embodiments the stem cell is a neural stem cell.

A screening assay is provided according to other aspects of the invention. The assay involves obtaining a biological sample from a subject associated with Down Syndrome, and identifying mitochondrial deletion that is present in the biological sample but not in a normal biological sample, wherein the mitochondrial deletion is predictive of Down Syndrome in a fetus of the subject associated with Down Syndrome.

In some embodiments the subject associated with Down Syndrome is a subject who has carried a fetus known to have a chromosomal abnormality associated with Down Syndrome. In other embodiments the mitochondrial deletion is identified using a subtractive hybridization assay.

A kit for assessing mitochondrial status in a maternal sample, is provided in another aspect. The kit includes a reagent for detecting a mitochondrial deletion associated with altered metabolic activity, and instructions for utilizing the reagent to identify the deletion as a predictor of a pre-disposition to a chromosomal abnormality associated with Down Syndrome in a fetus. The kit optionally includes a collection device for collecting a sample of peripheral blood. The reagent may be a nucleic acid probe. The kit may also include a labeling system for labeling the nucleic acid probe.

A kit for assessing mitochondrial status in a maternal sample including a reagent for detecting a level of mitochondrial membrane potential and instructions for utilizing the reagent to identify the level of mitochondrial membrane potential as a predictor of a pre-disposition to a chromosomal abnormality associated with Down Syndrome in a fetus is also provided. In some embodiments the reagent is mitotracker dye.

In yet another aspect, the invention is a neural stem cell having an isolated UCP4 gene under the control of a promoter. In some embodiments the cell includes an isolated UCP2 gene under the control of a promoter.

According to another aspect the invention is a neural stem cell having an isolated UCP2 gene under the control of a promoter. In one embodiment the cell includes an isolated UCP4 gene under the control of a promoter.

In some embodiments the promoter is an inducible promoter.

A method of generating neural tissue comprising implanting the neural stem cell into a subject, inducing expression of the UCP2 gene to grow neural tissue, and inducing expression of the UCP4 gene to differentiate the neural stem cells into neural tissue is also provided.

The invention in other aspects is a modified oocyte or embryonic cell which is an oocyte or embryonic cell having a microinjected heterologous mitochondria.

Each of the limitations of the invention can encompass various embodiments of the invention. It is, therefore, anticipated that each of the limitations of the invention involving any one element or combinations of elements can be included in each method and product.

### BRIEF DESCRIPTION OF THE FIGURES

The present invention may be more easily and completely understood when taken in conjunction with the accompanying figures.

FIG. 1 is a graph depicting the presence of UCP in neuronal Stem Cells. C17.2 mouse neuronal stem cells stained with either Anti-UCP2 antibody or Anti-UCP4 as shown in the graphs of FIG. 1a (cell surface UCP) and 1b (intracellular UCP).

FIG. 2 is a graph depicting neuronal stem cells in response to H₂O₂ with increased B7 and Fas. C17.2 mouse neuronal stem cells were treated or not with H₂O₂ and stained with Anti-B71 (FIG. 2a) or Anti-Fas (CD95) (FIG. 2b) antibodies (Pharmingen).

FIG. 3 is a graph depicting assessment of Cell Death in Mouse Oligodendrocyte cells in response to H₂O₂. Mouse oligodendrocyte cells were pre-treated or not with H₂O₂ and then incubated with a higher concentration of H₂O₂ for an additional time frame followed by analysis for percent death both flow cytometrically (FIG. 3a) and by Trypan Blue Exclusion (FIG. 3b).

FIG. 4 is a graph depicting assessment of Cell Death, cell surface Fas, and mitotracker fluorescence in Mouse Oligodendrocyte cells in response to H₂O₂. Mouse oligodendrocyte cells were pre-treated or not with H₂O₂ and then were incubated with higher concentrations of H₂O₂ and assessed using Trypan Blue exclusion (FIG. 4a and FIG. 4b) and Mitotracker (FIG. 4e and FIG. 4f). The cells were harvested and stained with Anti-Fas (CD95) antibody (Pharmingen) as indicated. Expression of Fas was measured on both live (FIG. 4c) and dead cell (FIG. 4d) populations.

FIG. 5 is a graph depicting assessment of Cell Death, cell surface Fas, and mitotracker fluorescence in Rat pheochromocytoma cells in response to H₂O₂. Rat pheochromocytoma cells were pre-treated or not with H₂O₂ and then incubated with a higher concentration of H₂O₂ for an additional time frame. The cells were harvested and stained with Anti-Fas (CD95) antibody (Pharmingen) as indicated (FIGS. 5b and 5C). They were also analyzed for percent death flow cytometrically (FIG. 5a) and stained with the fluorescent probe MitoTracker Red (FIG. 5d).

### DETAILED DESCRIPTION

The invention, in some aspects, relates to methods and products relating to the diagnosis and treatment of Down Syndrome. It was discovered according to some aspects of the invention that the mitochondrial membrane potential and specific mitochondrial deletions are associated with chromosomal abnormalities resulting in Down Syndrome. It was discovered that mitochondrial membrane potential and/or mitochondrial deletions of maternal cells can be used to predict a predisposition to Down Syndrome in a fetus.

The invention described herein demonstrates an intimate connection between cellular energetics and cell survival and growth. The energy metabolism of a cell is a key factor for determining cellular fate i.e., how the immune system interacts with that cell. In cells there are a limited number of metabolic states, depending on the fuel the cell consumes. These include glucose (carbohydrates), lipids (fats), and proteins. In particular, it has been discovered that whereas the ability to efficiently use fat for fuel in normal cells confers healthy stable cells the same metabolic process in subjects having Down Syndrome results in neural degeneration. Uncoupling proteins play an important role in this mechanism because they are instrumental in the fat burning process. As a result, changes in metabolism (caused by stresses, fuel availability, age, hormones, radiation, drugs, etc.) can have detrimental effects in subjects having Down Syndrome.

It has been discovered that changes in intracellular metabolism that occur in the Ts65Dn mouse model of trisomy of chromosome 21 affect susceptibility of neurons to immune mediated death. Mitochondrial deletions alter critical metabolic pathways and these alterations, in turn, directly affect susceptibility of neural cells to disease and death i.e., Fas (CD95)-induced death. It has been discovered that stimulating the cell surface molecule Fas (CD95) on peripheral sensory neurons can induce rapid, extensive neurite outgrowth in vitro and can accelerate functional recovery in vivo after sciatic nerve crush injury. Fas engagement on the neurons is likely providing a survival signal that is either lost or dysfunctional in people with Down Syndrome during development. Our data shows that the consequence of Fas engagement can be affected by metabolic state of the developing neurons and can result in either accelerated regeneration or increased susceptibility to cell death as a function of the metabolic state and the environment of the neuron. Restoration of the normal Fas signal by metabolic intervention may induce survival and regeneration of defective neurons. We used neurons from Ts65Dn mouse model for Down Syndrome (Davisson, M. T., et al. 1993. Prog. Clin. Biol. Res. 383: 117) to conduct studies on the effect of metabolic changes in Down Syndrome.

The mitochondrial respiration system is an important source of intracellular reactive oxygen species and other free radicals. The levels of reactive oxygen intermediates (ROI) are increased in the Down Syndrome neurons and reduced mitochondrial redox state and membrane potential reflect impaired mitochondrial function. It has also been discovered that mutations in mitochondrial DNA (mtDNA) result in increases in free radicals and reduced ATP levels and that this mitochondrial dysfunction affects neuronal development and the pathogenesis of Down Syndrome.

In non-dividing cells mitochondria provide over 90% of cellular ATP. The details of this energy storage process are complex, but there are key parameters that control ATP production. These include a proton gradient across the inner mitochondrial membrane, electron transport along the inner membrane and respiratory complexes within the inner membrane. The membrane potential depends on the maintenance of a proton gradient across the inner mitochondrial membrane. Oxygen complexes are used to facilitate the electron flow. Thus normal by-products of energy production are reactive oxygen species.

Thus, the invention in some aspects relates to a screening or diagnostic method for identifying a subject that is pre-disposed to developing Down Syndrome. The method involves the assessment of mitochondrial membrane potential and/or mitochondrial deletions in a biological sample from the subject.

Down Syndrome is a congenital defect that produces a broad spectrum of physical abnormalities in a subject, including anomalies of the gastrointestinal tract, increased risk of leukemia, defects of the immune and endocrine systems, early onset of Alzheimer's dementia and distinct facial and physical features, and a rather severe mental retardation. The phenotypic consequences of Down Syndrome have been believed to result from the overexpression and subsequent interactions of a subset of chromosome 21 genes.

Definitive prenatal diagnosis of fetal chromosome abnormalities leading to Down's syndrome typically involves amniocentesis culturing. The procedure involves the aspiration of a small sample of amniotic fluid (amniocentesis), culturing of the fetal cells contained in the fluid, and determination of the karyotype of these cells and thus the fetus. Direct transcervical aspiration of chorionic villi (chorionic villus sampling, or CVS) has also been used for prenatal diagnosis. Both procedures are relatively safe and reliable, but do involve some risk, including risk of miscarriage, and, in the case of CVS, also risk of limb hypoplasia in babies born following the procedure. The major indications for the use of the diagnostic techniques for the detection of chromosome abnormalities are maternal age (usually offered to all mothers over the age of 35 at the time of expected delivery), the presence of a parental chromosome abnormality, or a maternal history of carrying a previous trisomic child or aborted fetus karyotyped to be trisomic.

Amniocentesis is the most common invasive prenatal diagnostic procedure. In amniocentesis, amniotic fluid is sampled by inserting a hollow needle through the mother's anterior abdominal and uterine walls into the amniotic cavity by piercing the chorion and amnion. It is usually performed in the second trimester of pregnancy. CVS is performed primarily during the first trimester, and involves collecting cells from the chorion which develops into the placenta. Another invasive prenatal diagnostic technique is cordocentesis or percutaneous umbilical cord blood sampling, commonly known as fetal blood sampling. Fetal blood sampling involves obtaining fetal blood cells from vessels of the umbilical cord, and is often performed about the 20th gestational week.

The triple marker test has been used to screen for Down Syndrome pregnancies. It combines maternal age with serum measurements of hCG, α-fetoprotein, and unconjugated estriol (Bogart, M. H., et al., Prenat. Diagn. 7:623-630 (1987), U.S. Pat. No. 4,874,693 to Bogart, Wald, N. J., et al., Br. J. Obstet. Gynaecol. 95: 334-341 (1988), and Canick, J. A., J. Clin. Immunoassay 13: 30-33 (1990)). More recently, serum-free β-subunit tests and free β-subunit-α-fetoprotein combinations have been introduced as alternative Down Syndrome-screening methods (Macri, J. N., et al., Am. J. Obstet. Gynecol. 163: 1248-1253 (1990) and Spencer, K., et al., Ann. Clin. Biochem. 30: 394-401 (1993)). The best serum free β-subunit combination, or the optimal triple marker test, however, detects only 60 to 65 percent of Down's syndrome cases, with a 5 percent false-positive rate. Such rates mean that the double and triple screens still fail to detect a significant number of Down Syndrome affected pregnancies. The methods of the invention provide an alternative screening method that is minimally or non-invasive.

It has been discovered according to the invention that Down Syndrome is actually associated with metabolic mitochondrial changes that can be detected in maternal samples and are predictive of fetal Down Syndrome, and also that correction of such deletions and changes can be used therapeutically.

The methods of the invention are useful in subjects. A subject as used herein means vertebrates such as humans, primates, horses, cows, pigs, sheep, goats, dogs, cats and rodents. Generally the subject is a maternal subject. A maternal subject as used herein refers to a female subject.

The maternal subject may be pregnant or not. For instance, it may be desirable to assess the risk in a subject of conceiving a fetus that has Down Syndrome prior to the conception. If a subject is identified as having a high risk of conceiving a child with Down Syndrome then the subject may choose to undertake a therapeutic measure to avoid conceiving a child with Down Syndrome. One therapeutic intervention that the subject could undertake involves the mitochondrial replacement therapy described herein. Another method may involve isolation of oocyes and screening of particular oocytes for mitochondrial deletions or membrane potential prior to an in vitro fertilization (IVF) procedure.

Alternatively the subject may already be pregnant. Traditionally most screening tests or diagnostic tests occur during a pregnancy. Some tests are required to be performed after the fetus achieves a minimal gestational age. One advantage of the instant diagnostic/screening method is that it can be performed any time during the pregnancy, even during the first few days because the test is performed on the maternal sample.

The method involves detection of mitochondrial deletions that correlate with Down Syndrome. Mitochondrial deletions may be detected using routine methods known in the art. For instance, total or mitochondrial DNA may be isolated and probed using a procedure such as a Southern blot to identify known deletions. Other methods that could be used include PCR. For instance, long-extension PCR may be used to map mitochondrial DNA deletions. An example of this method is described in Liang et al Diabetes, v. 46, 1997, p 920, which is incorporated by reference.

The mitochondrial deletions that are useful for predicting Down Syndrome are those that play a role in regulating mitochondrial metabolism. For instance, several of these deletions are described in Liang et al. One example is a 4,977 bp deletion that occurs primarily in the complex I genes.

Additional deletions useful according to the methods of the invention may be identified by using a screening assay of the invention. The screening assay involves obtaining a biological sample from a subject associated with Down Syndrome, and identifying a mitochondrial deletion that is present in the biological sample but not in a normal biological sample. A subject associated with Down Syndrome is a subject that has been identified as having a high likelihood of conceiving a child with Down Syndrome. For instance, such a subject could be identified by having a known mitochondrial deletion, having carried a fetus known to have a chromosomal abnormality associated with Down Syndrome or an aborted fetus karyotyped to be trisomic. Such a screening assay can be performed using methods known in the art, such as a subtractive hybridization assay.

Membrane potential may be assessed by any method known for determining membrane potential. For instance membrane potential may be directly measured using flow cytometric experiments with Mitotracker Red dyes. Other methods involve detection of co-stimulatory molecules on the cell surface. As described below (and in co-pending application Ser. Nos. 09/277,575, 09/599,760 and 10/272,432) in more detail, changes in membrane potential are correlative with changes in cell surface molecules. Cell surface molecule expression can be assessed using antibodies or other labeling reagents.

Any biological sample containing cells from the subject can be employed in methods of the invention, including, but not limited to, serum, plasma, cheek cells, muscle, skin, and amniotic fluid. Plasma or serum are preferred because samples are more voluminous and sampling involves no risk of harm to the fetus and are relatively non-invasive to the mother.

The diagnostic/screening methods described herein provide a means to screen the population of pregnant women to determine which pregnancies are at risk for Down Syndrome and other serious genetic defects. The risk may be calculated based on the results of the screen alone or along with other cofactors, such as, maternal age, to determine if the risk is high enough to warrant an invasive diagnostic procedure, such as, amniocentesis, CVS or fetal blood sampling. These prenatal screens can be used either alone or in combination with other screening or diagnostic methods. Other screening methods include, but are not limited to, estriol measurements, hCG assays, β core fragment analyses, free β-subunit or free α-subunit analyses, PAPP-A or CA125 analyses, α-fetoprotein analyses, inhibin assays, triple screen, and ultrasound. Biochemical screening for neural tube defects may be accomplished by measuring alpha-fetoprotein (AFP). The triple screen measures AFP, human chorionic gonadotropin (hCG) and unconjugated estriol in the serum of pregnant women.

The invention also involves methods for modifying an oocyte or embryonic cell by microinjecting a heterologous mitochondria in order to stabilize mitochondrial membrane potential or overcome mitochondrial deletions. As described above, defects in mitochondria tend to accumulate with age and are associated with disorders such as Down Syndrome. In order to prevent a fetus from developing Down Syndrome an oocyte or embryo can be treated to replace the defective mitochondria.

One method of replacing the defective mitochondria is by microinjection. Mitochondria from healthy cells can be isolated and transferred to the oocyte or embryo. Mitochondria from young donors are generally healthy, but the mitochondria can be assessed by detection of mitochondrial membrane potential or for the presence of deletions to establish that they are healthy prior to transfer. Alternatively mitochondria may be isolated from cells of the recipient (i.e. maternal or embryonic cells) and then modified to become normal. These methods may be accomplished, for example, by overexpression of UCP in the cells to improve membrane potential (i.e., see co-pending applications described above) or correction or replacement of the defective genes in the mitochondrial DNA. Many methods of microinjection are known in the art. For example U.S. Pat. No. 5,877,008 described a microinjector for blastocysts. Many others are also known and can be used in the methods of the invention.

Another method for replacing the defective mitochondria involves manipulation of existing mitochondria. For instance the mitochondria of the oocyte or embryo may be manipulated to force expression (i.e. transfection of UCP) or upregulate UCP or correction or replacement of the defective mitochondrial DNA directly in the cells.

It is possible to inject the mitochondria directly into the oocyte or by using electroporation fusion. Such techniques are disclosed in Collas and Barnes, Mol. Reprod. Dev., 38:264-267 (1994), incorporated by reference in its entirety herein.

As used herein, the term “oocyte” refers to a female gamete cell and includes primary oocytes, secondary oocytes and mature, unfertilized ovum. An oocyte is a large cell having a large nucleus (i.e., the germinal vesicle) surrounded by ooplasm. The ooplasm contains non-nuclear cytoplasmic contents including mRNA, ribosomes, mitochondria, yolk proteins, etc. The membrane of the oocyte is referred to herein as the oocyte plasma membrane.

The term “pre-maturation oocyte” as used herein refers to a female gamete cell following the oogonia stage (i.e., mitotic proliferation has occurred) that is isolated from an ovary (e.g., by aspiration) but which has not been exposed to maturation medium in vitro. Those of skill in the art know that the process of aspiration causes oocytes to begin the maturation process but that completion of the maturation process (i.e., formation of a secondary oocyte which has extruded the first polar body) in vitro requires the exposure of the aspirated oocytes to maturation medium. Pre-maturation oocytes will generally be arrested at the first anaphase of meiosis.

The term “pre-fertilization oocyte” as used herein refers to a female gamete cell such as a pre-maturation oocyte following exposure to maturation medium in vitro but prior to exposure to sperm (i.e., matured but not fertilized). The pre-fertilization oocyte has completed the first meiotic division, has released the first polar body and lacks a nuclear membrane (the nuclear membrane will not reform until fertilization occurs; after fertilization, the second meiotic division occurs along with the extrusion of the second polar body and the formation of the male and female pronuclei). Pre-fertilization oocytes may also be referred to as matured oocytes at metaphase II of the second meiosis.

The terms “unfertilized egg” or “unfertilized oocyte” as used herein refers to any female gamete cell which has not been fertilized and these terms encompass both pre-maturation and pre-fertilization oocytes.

As used herein, the term “egg” when used in reference to a mammalian egg, means an oocyte surrounded by a zona-pellucida and a mass of cumulus cells (follicle cells) with their associated proteoglycan. The term “egg” is used in reference to eggs recovered from antral follicles in an ovary (these eggs comprise pre-maturation oocytes) as well as to eggs which have been released from an antral follicle (a ruptured follicle). The mature eggs are removed from the ovary transvaginally using a needle, preferably guided under ultrasound.

After the oocyte is subjected to conditions to improve mitochondria the oocyte is fertilized. The fertilization is performed in a manner known per se, either by standard in vitro fertilization (IVF) or by intracytoplasmic sperm injection (ICSI), for example, as described in an overview article by Davis & Rosenwaks (in Reproductive, Endocrinology, Surgery and Technology Chapter 124, pp. 2319-2334, Editors: Adashi, Rock and Rosenwaks, 1995, Lippencott-Raven publishers). After the oocyte is fertilized, the zygote is allowed to develop a few days in culture and is subsequently transferred to the uterus of the patient or cryopreserved.

The fertilized oocytes may be first microinjected with mitochondria by standard techniques. Alternatively, they may be cultured in vitro until a “pre-implantation embryo” is obtained, which can be microinjected or otherwise manipulated. Such pre-implantation embryos preferably contain approximately 16 to 150 cells. The 16 to 32 cell stage of an embryo is commonly referred to as a morula. Those pre-implantation embryos containing more than 32 cells are commonly referred to as blastocysts. They are generally characterized as demonstrating the development of a blastocoel cavity typically at the 64 cell stage. Methods for culturing fertilized oocytes to the pre-implantation stage include those described by Gordon, et al. (1984) Methods in Enzymology 101:414; Hogan, et al. (1986) in Manipulating the Mouse Embryo, Cold Spring Harbor Laboratory Press, Cold Spring Harbor, N.Y. (for the mouse embryo); and Hammer, et al. (1985) Nature 315:680 (for rabbit and porcine embryos) Gandolfi, et al. (1987) J. Reprod. Fert. 81:23-28; Rexroad, et al. (1988) J. Anim. Sci. 66:947-953 (for ovine embryos) and Eyestone, W. H. et al. (1989) J. Reprod. Pert. 85:715-720; Camous., et al. (1984) J. Reprod. Fert. 72:779-785; and Heyman, Y., et al. (1987) Theriogenology 27:5968 (for bovine embryos). Such pre-implantation embryos are thereafter transferred to the subject.

The invention also encompasses methods for promoting tissue generation. Tissue generation as used herein refers to the induction of differentiation and or growth. For instance, stem cells may be treated by the methods of the invention to be susceptible to growth and differentiation conditions to promote cell generation, i.e. by microinjection of a healthy mitochondria. Such cells can differentiate into mature differentiated cells under the appropriate conditions. Additionally, tissue generation refers to the proliferation of cells, such as organ tissue, when it is desirable to generate new or repair existing organs.

The methods are useful, for instance, when a stem cell source has a damaged or defective mitochondria. It is particularly useful when the stem cell source is autologous to the recipient. The methods of the invention enable the use of autologous tissue for repair of damaged tissue even when the stem cells have defective mitochondria. The methods are achieved by microinjecting mitochondria into the stem cell to repair the metabolic function of the cell. The modified stem cells can then be used in the repair of injured or diseased tissue.

Once the stem cell is modified by the addition of the autologous or heterologous mitochondria, the stem cell can be subjected to growth or quiescent conditions. Such conditions are described in co-pending application Ser. Nos. 09/277,575, 09/599,760 and 10/272,432, having common inventorship. It was recognized that the choice of fuel (e.g., glucose and/or lipid) for mitochondrial metabolism is part of a metabolic behavior that regulates the interaction of the cell with any other cell including cells of the immune system. There are at least three metabolic base states and these base states are defined by the levels of reactive oxygen inside the cell. The levels of reactive oxygen impact whether a tissue is ignored by the immune system (a growth inhibited state), the tissue undergoes regenerative growth nurtured by the immune system (a growth induced state), or the tissue is sensitive to immune induced death i.e. as would happen to infected or severely damaged cells (an immune targeted state).

Cells in the immune targeted state have high intracellular levels of reactive oxygen. Under these conditions co-stimulatory molecules are expressed under conditions which lead to rejection of the tissue. For instance, high levels of intracellular reactive oxygen induced under conditions in which no additional metabolic strategy can deal with it produce cells in the immune targeted state that can be targeted for destruction. For example, uncoupling proteins cannot be effectively expressed, the expression of uncoupling proteins has been disabled by drugs which interrupt UCP expression or activity such as anti-sense to UCP, or the uncoupled, protective metabolic state has been negatively affected by metabolic interference from such compounds as chemotherapeutic agents (i.e., adriamycin, 5FU, methotrexate, trimetrexate, cisplatin, etc. at concentrations greater than 10-8 M in vivo), radiation of any kind at levels greater that 25 to 30 grey, high intensity, high frequency microwaves, gamma radiation above 25 grey. Additionally, conditions which disable other protective strategies, such as manganese or copper/zinc superoxide dismutase, glutathione-S reductase, etc. (i.e. inhibitors of such compounds) could tip the balance to a metabolic strategy in which the levels of reactive oxygen are high enough to trigger destructive immune recognition, particularly in the absence of growth signals which tip the balance towards the growth induced state. Another example of conditions causing high reactive oxygen leading to the immune targeted state involves a combined approach of two strategies resulting in high intracellular reactive oxygen such as, for example, lower level radiation (10 to 25 grey) with less that 10-8 M chemotherapeutic.

Cells in the growth induced state have intermediate levels of intracellular reactive oxygen causing an induction in co-stimulatory molecules. These cells are maintained, preferably during exposure to the immune system, under growth conditions, such that the cells are encouraged to grow. Growth promoting conditions include but are not limited to the following: insulin (e.g., for modulation growth of brain, eye, skin, muscle, kidney, etc); nerve growth factor; fibroblast growth factor (e.g., for modulating growth of connective tissues); platelet derived growth factor (e.g., for modulating growth of platelets); erythropoietin (e.g., for modulating red blood generation); and cytokines including, for example, IL-2, IL-4, γ interferon, α and β interferons, TNF (tumor necrosis factor) α, TGF (T-cell growth factor) α and β, and lymphotoxin.

Thus methods for producing cells in the growth induced state involve generating an intermediate level of reactive oxygen under growth conditions. These moderate levels of intracellular reactive oxygen produced by growth conditions prime the cells for repair.

Cells in the growth inhibited state are immune-privileged cells. These cells are maintained under conditions in which lipids are preferentially used for fuel. The cells have lower mitochondrial membrane potential, are less likely to have surface MHC, are less easily damaged by free radicals, and have relatively lower levels of (or no) co stimulatory molecule expression. Cells in this state are not recognized by the immune system.

Induced repair and regeneration of tissues is important in many contexts and can be achieved by causing the cells to assume a growth induced state. For instance, regeneration of neurons is most important in helping stroke victims or people with spinal cord injuries.

Another aspect of the invention is a method for reinnervating an injured tissue. The method involves the step of microinjecting a mitochondria into a neural stem cell and growing the neural stem cell under conditions to promote growth and differentiation to reinnervate the injured tissue. The nerve cell may be treated in vivo or may be manipulated in vitro and then transplanted. Methods are known in the art for implanting nerve cells into living tissue. For example, nerves can be implanted directly into exposed tissue or may be implanted in biodegradable tubes which will guide the extension of the nerve into surrounding tissue where it can be differentiated.

An injured nerve tissue is a tissue in which nerve damage has been sustained. An injured tissue may include for example, an injured spinal chord, a severed or severely damaged limb or any other tissue which can be innervated and in which the nerve has been damaged. Conditions involving injuries such as brain ischemia, spinal chord damage, and severance of limbs often causes extensive neuronal cell death. When a nerve is severed, the regions of the nerve cells which are distal to the severance become separated from the nerve cell body and degenerate. After such a severance, it is possible for the nerve cell body to regenerate by re-extension of the severed axons. This process of nerve regeneration does not occur naturally in the absence of certain environmental conditions.

The invention also includes a method for treating a neurodegenerative disorder. A “neurodegenerative disorder” as used herein, is a disorder associated with the death or injury of neuronal cells. For example, the loss of dopaminergic neurons in the substantia nigra ultimately leads to Parkinson's Disease. The deposition of β-amyloid protein in the brain generally causes neural damage leading to Alzheimer's Disease. These diseases, which include Alzheimer's Disease, Multiple Sclerosis (MS), Huntington's Disease, Amyotrophic Lateral Sclerosis, and Parkinson's Disease, have been linked to the degeneration of neural cells in particular locations of the CNS, leading to the inability of these cells or the brain region to carry out their intended function.

The invention also relates to methods for facilitating repair or generating other types of tissue for transplantation or in vivo methods, such as wound healing or tissue growth.

A stem cell is a cell that has the ability to exhibit self-renewal or to generate more of itself, i.e., a cell with the capacity for self-maintenance. Generally stem cells are capable of proliferation, self-maintenance, and the production of a large number of differentiated functional progeny. The role of stem cells is to replace cells that are lost by natural cell death, injury or disease. The presence of stem cells in a particular type of tissue usually correlates with tissues that have a high turnover of cells. A neural stem cell is an undifferentiated neural cell.

Stem cells can be used for transplantation into a heterologous, autologous, or xenogeneic host. Multipotent stem cells can be obtained from embryonic, post-natal, juvenile or adult tissue. The tissue can be obtained from any animal source. A preferred source of tissue is from mammals, preferably rodents and primates, and most preferably, mice and humans.

In the case of a heterologous donor animal, the animal may be euthanized, and the neural tissue and specific area of interest removed using a sterile procedure. Areas of particular interest include any area from which neural stem cells can be obtained that will serve to restore function to a degenerated area of the host's nervous system. Human heterologous neural stem cells may be derived from fetal tissue following elective abortion, or from a post-natal, juvenile or adult organ donor. Autologous neural tissue can be obtained by biopsy, or from patients undergoing neurosurgery in which neural tissue is removed, for example, during epilepsy surgery, temporal lobectomies and hippocampalectomies. Neural stem cells have been isolated from a variety of adult CNS ventricular regions, including the frontal lobe, conus medullaris, thoracic spinal cord, brain stem, and hypothalamus.

It is well recognized in the art that transplantation of tissue into the CNS offers the potential for treatment of neurodegenerative disorders and CNS damage due to injury (review: Lindvall, (1991) Tins vol. 14(8): 376-383). Transplantation of new cells into the damaged CNS has the potential to repair damaged circuitries and provide neurotransmitters thereby restoring neurological function. Transplantation can be accomplished by administering cells to the particular region of the subject using any method which maintains the integrity of surrounding tissues, i.e., by injection cannula. Injection methods exemplified by those used by Duncan et al. J. Neurocytology, 17:351-361 (1988), and scaled up and modified for use in humans are useful. Additional approaches and methods may be found in Neural Grafting in the Mammalian CNS, Bjorklund and Stenevi, eds., (1985).

Neural stem cells when administered to the particular neural region preferably form a neural graft, wherein the neuronal cells form normal neuronal or synaptic connections with neighboring neurons, and maintain contact with transplanted or existing glial cells which may form myelin sheaths around the neurons' axons, and provide a trophic influence for the neurons. As these transplanted cells form connections, they re-establish the neuronal networks which have been damaged due to disease and aging.

Survival of the graft in the living host can be examined using various non-invasive scans such as computerized axial tomography (CAT scan or CT scan), nuclear magnetic resonance or magnetic resonance imaging (NMR or MRI) or more preferably positron emission tomography (PET) scans. Functional integration of the graft into the host's neural tissue also can be assessed by examining the effectiveness of grafts on restoring various functions, including but not limited to tests for endocrine, motor, cognitive and sensory functions. Motor tests which can be used include those which quantitate rotational movement away from the degenerated side of the brain, and those which quantitate slowness of movement, balance, coordination, akinesia or lack of movement, rigidity and tremors. Cognitive tests include various tests of ability to perform everyday tasks, as well as various memory tests, including maze performance.

Modified neural stem cells may also be generated using UCP constructs. An isolated UCP gene under the control of a promoter may be transfected into neural stem cells to produce a population of cells that can be tightly controlled for the process of tissue generation. It has been discovered herein that neural stem cells express UCP2 during a cellular division phase. When the cells stop dividing and differentiate the expression of UCP2 is turned off and the expression of UCP4 is induced. UCP2 and UCP4 constructs can be utilized to control the growth and differentiation of the cells. For instance, a neural stem cell can be transfected with a UCP2 and/or UCP4 construct that can be activated to express either of the UCPs depending on whether growth or differentiation is desirable. It may be desirable to control the population of neural cells so that they are in a growth phase until an adequate amount of tissue is generated. Then the cells can be induced to differentiate using the UCP4. The UCP2 and UCP4 can be part of a single construct or separate constructs. Optionally they can be under the control of inducible promoters.

An isolated molecule is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide, or polysaccharide. Because an isolated molecular species of the invention may be admixed with a pharmaceutically-acceptable carrier in a pharmaceutical preparation, the molecular species may comprise only a small percentage by weight of the preparation. The molecular species is nonetheless substantially pure in that it has been substantially separated from the substances with which it may be associated in living systems.

The UCP nucleic acid can be delivered to a cell such that a peptide encoded for by the nucleic acid will be expressed in a cell in order to produce cells or reagents useful according to the invention. These methods may be accomplished using expression vectors which are prepared and inserted into cells using routine procedures known in the art. These procedures are described in more detail in co-pending patent application U.S. Ser. No. 09/277,575, having common inventorship, which is hereby incorporated by reference. Nucleic acids encoding UCP are known in the art and may be found in many references as well as in genbank under various accession numbers. The nucleic acid used will depend on the purpose of generating the expression vector useful in the methods of the invention. Those of skill in the art will be able to select the appropriate nucleic acid for expression. For instance, when it is desirable to express UCP2 in a mitochondria of a cell to promote uncoupling of the mitochondria, any of the UCP2 nucleic acids may be selected. Human UCP2 may be a preferred nucleic acid. Human UCP2 is described for instance in ATCC accession numbers BC011737, NM_{—}003355, U76367, and AF306570. UCP4 is described for instance in ATCC accession numbers AF110532, BC063945, NM_{—}053500, AY358711, and AB106930.

The nucleic acids useful herein may be operably linked to a gene expression sequence which directs the expression of the nucleic acid within a eukaryotic cell. The “gene expression sequence” is any regulatory nucleotide sequence, such as a promoter sequence or promoter-enhancer combination, which facilitates the efficient transcription and translation of the nucleic acid to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPTR), adenosine deaminase, pyruvate kinase, and actin. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined nucleic acid. The gene expression sequences optionally include enhancer sequences or upstream activator sequences as desired.

The nucleic acid sequence and the gene expression sequence are said to be “operably linked” when they are covalently linked in such a way as to place the transcription and/or translation of the coding sequence under the influence or control of the gene expression sequence. If it is desired that the sequence be translated into a functional protein, two DNA sequences are said to be operably linked if induction of a promoter in the 5′ gene expression sequence results in the transcription of the sequence and if the nature of the linkage between the two DNA sequences does not (1) result in the introduction of a frame-shift mutation, (2) interfere with the ability of the promoter region to direct the transcription of the sequence, or (3) interfere with the ability of the corresponding RNA transcript to be translated into a protein. Thus, a gene expression sequence would be operably linked to a nucleic acid sequence if the gene expression sequence were capable of effecting transcription of that nucleic acid sequence such that the resulting transcript might be translated into the desired protein or polypeptide.

The terms “treat” and “treating” as used herein refer to preventing the development of a disease, reducing the symptoms of disease, and/or inhibiting the progression of a disease, such as Down Syndrome.

The invention involves in vitro, in vivo, and ex vivo technologies. The in vitro methods of the invention are useful for a variety of purposes. For instance, the methods of the invention may be useful for testing putative therapeutics on cells (i.e. H1b or HTk cells) cultured in vitro as well as the diagnostics described herein.

In addition to the in vitro methods, the methods of the invention may be performed in vivo or ex vivo in a subject to manipulate one or more cell types within the subject. An “ex vivo” method as used herein is a method which involves isolation of a cell from a subject, manipulation of the cell outside of the body, and reimplantation of the manipulated cell into the subject. The ex vivo procedure may be used on autologous or heterologous cells. In some embodiments, the ex vivo method is performed on cells that are isolated from bodily fluids such as peripheral blood or bone marrow, but may be isolated from any source of cells. When returned to the subject, the manipulated cell will have a microinjected mitochondria. Ex vivo manipulation of cells has been described in several references in the art, including Engleman, E. G., 1997, Cytotechnology, 25:1; Van Schooten, W., et al., 1997, Molecular Medicine Today, June, 255; Steinman, R. M., 1996, Experimental Hematology, 24, 849; and Gluckman, J. C., 1997, Cytokines, Cellular and Molecular Therapy, 3:187. In vivo methods are also well known in the art. The invention thus is useful for therapeutic purposes and also is useful for research purposes such as testing in animal or in vitro models of medical, physiological or metabolic pathways or conditions.

The compositions useful in the invention may be formulated or unformulated. In general, the delivery formulations useful in the invention include colloidal dispersion systems, carriers, biological vectors, and any other type of formulation known in the art.

As used herein, a “colloidal dispersion system” refers to a natural or synthetic molecule, other than those derived from bacteriological or viral sources, capable of delivering to and releasing the composition in a subject. Colloidal dispersion systems include macromolecular complexes, nanocapsules, microspheres, beads, and lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0μ can encapsulate large macromolecules within the aqueous interior and these macromolecules can be delivered to cells in a biologically active form (Fraley, et al., Trends Biochem. Sci., 6:77 (1981)).

Lipid formulations for transfection are commercially available from QIAGEN, for example as EFFECTENE™ (a non-liposomal lipid with a special DNA condensing enhancer) and SUPER-FECT™ (a novel acting dendrimeric technology) as well as Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes were described in a review article by Gregoriadis, G., Trends in Biotechnology 3:235-241 (1985), which is hereby incorporated by reference.

In one particular embodiment, the preferred vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”, claiming priority to U.S. patent application serial no. 213,668, filed Mar. 15, 1994). PCT/US/0307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the compositions of the invention described herein are encapsulated or dispersed within the biocompatible, preferably biodegradable polymeric matrix disclosed in PCT/US/03307.

The polymeric matrix preferably is in the form of a microparticle such as a microsphere (wherein the composition is dispersed throughout a solid polymeric matrix) or a microcapsule (wherein the composition is stored in the core of a polymeric shell). Other forms of the polymeric matrix for containing the composition include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and composition are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time.

In another embodiment the delivery vehicle or vector is a biocompatible microsphere that is suitable for oral delivery. Such microspheres are disclosed in Chickering et al., Biotech. And Bioeng., (1996) 52:96-101 and Mathiowitz et al., Nature, (1997) 386:410-414.

It is also envisioned that certain compounds useful in the invention may be delivered to the subject in a biological vector which is a nucleic acid molecule which encodes for a particular protein, such as UCP that is desirable to express in vivo. The nucleic acid encoding the protein is operatively linked to a gene expression sequence which directs the expression of the nucleic acid within a eukaryotic cell, as described above.

Compaction agents also can be used alone, or in combination with, a vector of the invention. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver the compositions in a form that is more efficiently taken up by the cell or, more preferably, in combination with one or more of the above-described vectors.

Other exemplary compositions that can be used to facilitate uptake by a target cell of the compositions of the invention include calcium phosphate and other chemical mediators of intracellular transport, microinjection compositions, electroporation and homologous recombination compositions (e.g., for integrating a composition of the invention into a preselected location within the target cell chromosome).

The pharmaceutical preparations of the invention are administered to subjects in effective amounts. An effective amount means that amount necessary to delay the onset of inhibit the progression of, halt altogether the onset or progression of or diagnose the particular condition being treated. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

Generally, doses of active compounds will be from about 0.01 mg/kg per day to 1000 mg/kg per day. It is expected that doses range of 50-500 mg/kg will be suitable, in one or several administrations per day. In the event that a response in a subject is insufficient at the initial doses applied, higher doses (or effectively higher doses by a different, more localized delivery route) may be employed to the extent that patient tolerance permits. Multiple doses per day are contemplated to achieve appropriate levels of compounds.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. As used herein, the compositions of the invention may include various salts.

The compositions of the invention may be combined, optionally, with a pharmaceutically-acceptable carrier. The term “pharmaceutically-acceptable carrier” as used herein means one or more compatible solid or liquid filler, diluents or encapsulating substances which are suitable for administration into a human or other animal. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

Compositions suitable for parenteral administration conveniently comprise a sterile aqueous preparation of the compositions of the invention, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

A variety of administration routes are available. The particular mode selected will depend of course, upon the particular drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, pulmonary, sublingual, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule.

The pharmaceutical compositions may conveniently be presented in unit dosage form and may be prepared by any of the methods well-known in the art of pharmacy. All methods include the step of bringing the compositions of the invention into association with a carrier which constitutes one or more accessory ingredients. In general, the compositions are prepared by uniformly and intimately bringing the compositions of the invention into association with a liquid carrier, a finely divided solid carrier, or both, and then, if necessary, shaping the product.

Compositions suitable for oral administration may be presented as discrete units, such as capsules, tablets, lozenges, each containing a predetermined amount of the compositions of the invention. Other compositions include suspensions in aqueous liquids or non-aqueous liquids such as a syrup, elixir or an emulsion.

Other delivery systems, such as the vectors and delivery formulations described above may be used. One preferred delivery system can include time-release, delayed release or sustained release delivery systems. Such systems can avoid repeated administrations of the compositions of the invention described above, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the compositions of the invention is contained in a form within a matrix such as those described in U.S. Pat. Nos. 4,452,775, 4,667,014, 4,748,034 and 5,239,660 and (b) difusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

### EXAMPLES

Our previous work has focused on the regulation of Fas-induced apoptosis versus Fas-mediated proliferation (Desbarats, J. et al., 1998, Nat. Med., 4:1377; Desbarats, J. et al., 1999, PNAS, 96:8104; Desbarats, J et al., 2000, Nat. Med., 6:920; Desbarats, J. et al., 1996, PNAS, 93:11014). In T cells, we have shown that Fas engagement results in apoptosis in the absence of cytokines, whereas Fas engagement results in accelerated proliferation in the presence of the appropriate cytokines and their receptors (Desbarats, J. et al., 1999, PNAS, 96:8104). In the liver, we showed that Fas-mediated apoptosis could be prevented by surgically inducing a regenerative response (Desbarats, J. et al., 2000, Nat. Med., 6:920).

We have shown that engaging Fas on sensory neurons induces a rapid, extensive regenerative response. We used the dorsal root ganglia (DRG) neurite outgrowth assay to determine whether Fas engagement could induce axon regeneration in vitro. Analysis by flow cytometry revealed that DRG neurons express Fas. We cultured DRGs with anti-Fas antibodies, with isotype matched control antibodies (as a negative control) or with NGF (as a positive control). We found that anti-Fas antibody induced robust neurite outgrowth in DRGs. Anti-Fas antibody failed to induce neurite outgrowth in DRGs from lpr mice, which bear a mutation resulting in low to absent Fas expression. We also confirmed that DRGs from lpr mice showed delayed and diminished neurite outgrowth, even in the presence of NGF.

Addition of neutralizing anti-NGF antibodies completely inhibited NGF-induced neurite outgrowth. However, anti-Fas-induced neurite outgrowth may be independent of endogenous NGF. It is possible that anti-Fas antibody is blocking an endogenous inhibitory Fas/Fas ligand interaction, thus un-inhibiting the system and allowing regeneration. To exclude this possibility, we cultured DRGs with anti-Fas Ligand antibodies or with FasFc. FasFc is a construct consisting of soluble Fas bound to the Fc portion of IgG, and has been shown to block endogenous Fas/Fas ligand interactions by binding to Fas ligand. However, Fas Fc had no detectable effect on neurite outgrowth.

### General Methods:

Cell Culture. All tumor cells were grown in culture in complete RPMI medium (supplemented with 5% Fetal calf serum, glutamine, beta-mercapto-ethanol, antibiotics).

Flow Cytometry. Cells were harvested, counted, and resuspended at 10⁶ cells/100 μl in preparation for flow cytometric analysis. Cells were stained for intracellular H₂O₂ using 6-carboxy-2′,7′-dichlorodihydrofluorescein diacetate (DCF-DA, Molecular Probes, Eugene, Oreg.). Briefly, cells were incubated with 1 mM DCF-DA for 20 minutes, washed twice in PBS containing 5% fetal calf serum and analyzed flow cytometrically. Mitochondrial membrane potential was assessed using Mitotracker Red (CM-H₂XROS, Molecular Probes, Eugene, Oreg.). The cells were resuspended in warm (37° C.) PBS containing a final concentration of 0.5 micromolar dye. The cells were incubated for 30 minutes, pelleted, and resuspended in prewarmed medium for analysis. Data were acquired on a Coulter Elite Epics or Excel flow cytometer (Coulter, Hialeah, Fla.) and analyzed with CellQuest software, (Becton Dickinson, San Jose, Calif.). The Coulter Epics Elite flow cytometer has a single excitation wavelength (488 nm) and band filters for PE (575 nm), FITC (525 nm) and Red613 (613 nm) that was used to analyze the stained cells. Each sample population was classified for cell size (forward scatter) and complexity (side scatter), gated on a population of interest and evaluated using 40,000 cells. Each figure describing flow cytometric data represents one of at least four replicate experiments.

Cell Culture

All cell lines were cultured in RPMI 1640 culture medium. The medium is supplemented with 5% fetal bovine serum (FBS), 2 mM L-Glutamine, 500 units/mL pennicillin/500 μg/mL of streptomycin, 10 mM HEPES Buffer, 10⁻⁵M 2-mercaptoethanol (2-ME), 1 mM MEM Sodium Pyruvate, and 0.04 μg/mL of Gentamicin (All reagents from Gibco BRL). Cells were maintained at 37° C. in a humidified atmosphere under 5% CO₂ in air.

Cell Counting

Cells were harvested and resuspended in 1 mL of RPMI medium. A 1:20 dilution of the cell suspension was made by using 50 μL of trypan blue (Sigma chemicals), 45 μL of Phosphate Buffered Saline (PBS) supplemented with 2% FBS, and 5 μL of the cell suspension. Live cells were counted using a hemacytometer and the following calculation was used to determine cell number: Average # of Cells×Dilution×10⁴.

Preparation of Cell for Staining

For staining protocols, between 0.5×10⁶ and 1.0×10⁶ cells were used; all staining was done in a 96-well U-bottom staining plate. Cells were harvested by centrifugation for 5 minutes at 300×g, washed with PBS/2% FBS, and resuspended into PBS/2% FBS for staining. Cells were plated into wells of a labeled 96-well plate in 100 μL of PBS/2% FBS.

Cell Surface Staining

Non-permeabilized cells were stained with antibodies to the cell surface receptors Fas (CD95) (Pharmingen) or with antibodies to uncoupling proteins (anti-UCP2 antibody) (Alpha Diagnostic International). Antibodies for both the isotype control and actual stain were added to the cell suspension, mixed, and then placed on ice for and incubation of 25 minutes in the dark. Subsequently the cells were centrifuged at 300×g for 5 minutes and the supernatant removed. The cells were washed one time with 100 μL of PBS/2% FBS and then transferred into flow cyotmetric tubes containing 500 μL of PBS/2% FBS for analysis.

Intracellular Staining

Cells were prepared as described above. Cell membranes were then permeabilized using the Cytofix/Cytoperm kit (Pharmagin). 100 μL of Cytofix solution was added to all the cell suspensions and mixed well. This was placed on ice for an incubation time of 30 minutes in darkness. The cells were then washed twice with 100 μL of 1× PermWash buffer and then resuspended into 100 μL of 1× PermWash buffer for staining. Cells were stained according to the cell surface staining protocol. After staining and washing, cells were transferred into flow cytometric tubes containing 500 μL of PBS/2% FBS for analysis.

Metabolic Activity Assay

Cells were prepared as previously described. The specific metabolic dye was added and mixed into the cell suspension. This plate was then placed into the 37° incubator for a 20-minute incubation. After incubation, the cells were centrifuged at 300×g for 5 minutes, and the supernatant was removed. The cells were then washed once with PBS/2% FBS and transferred into flow cytometric tubes containing 500 μL of PBS/2% FBS for analysis

MitoTracker Red CM-H₂XROS (Molecular Probes): One vial of MitoTracker Red provides 10 tests and contains 50 μg. This is unstable and can't be stored. Therefore, when each vial was opened, 43 μL of Dimethysulfoxide (DMSO) was added. Once this was mixed well, 4.0 μL of this was added to each well containing the cells to be tested for their mitochondrial membrane potential. MitoTracker was used at a final concentration of 46 ng for each test.

5-(and -6)-Chloromethyl-2′,7′-dichlorodihydrofluorescein diacetate (CMH₂DCFDA) (Molecular Probes): One vial of DCFda provides 10 tests and contains 50 μg. This is unstable and can't be stored. Therefore, each vial was opened and 43 μL of DMSO was added. Once this was mixed well, 4.0 μL of this was added to each well containing the cells to be tested. DCFDA was used at a final concentration of 46 ng for each test.

LysoSensor Green DND-189 (Molecular Probes): LysoSensor is provided at a concentration of 1 mM in 50 μL of DMSO. The LysoSensor was thawed to room temperature immediately before use and 0.5 μL was added to each well containing the cells to be tested. LysoSensor was used at a final concentration of 5 nM for each test.

### Flow Cytometry

Once samples had been prepared and transferred into flow cytometric tubes, they were analyzed on a Becton/Dickinson Flow Cytometer. For antibodies that are PE conjugated and for MitoTracker Red, a program for red colored fluorochromes was utilized. For antibodies that are FITC conjugated and for DCFda, LysoSensor, and LysoTracker a program for green colored fluorochromes was used.
Statistical Analysis. Percents, and Geometric Mean Values

Percents: Gating is a tool provided by Cell Quest software and allows for the analysis of a certain population of cells. Gating around both the live and dead cell populations gave a percent of the cell numbers that was in each population. After the gates were drawn, a percent value of dead cells was calculated by taking the number of dead cells divided by the number of total cells and multiplying by one hundred.

Standard Error: When experiments were done in triplicate, a standard error of the mean value was determined using the Excel program (Microsoft). This identified the value given for the error bars seen on some figures.

Geometric Mean Fluorescence: When analyzing data on Cell Quest software, a geometric mean value will be given for each histogram plotted. Once the stained sample was plotted against the control (isotype or unstained), geometric mean fluorescence values were obtained for both histogram peaks. The stained control sample value was subtracted from sample to identify the actual fluorescence of the stained sample over that of the control.

### Example 1

### Role of Fas in Regulating Neural Generation

SH-SY5Y neuroblastoma cells are insensitive to Fas-mediated apoptosis. We used the well-characterized Fas-positive human neuroblastoma cell line SH-SY5Y to investigate Fas signaling in neuronal cells. Because Fas is best known as an inducer of apoptosis, we began by examining anti-Fas-treated SH-SY5Y cells for evidence of Fas-induced apoptosis. Jurkat cells, a human T cell leukemia line considered a model for Fas-mediated apoptosis (Wilson, D. et al., 1999, Cell Immunol., 194:67), provided a positive control. We confirmed that SH-SY5Y cells expressed cell surface Fas by flow cytometry. We then performed cell cycle analysis by flow cytometry on untreated and anti-Fas-treated Jurkat and SH-SY5Y cells after 48 hours in culture.

As previously reported, Jurkat T cells underwent Fas-induced apoptosis (7.6±2.8% apoptotic cells in the untreated cultures versus 31.3 f 3.5% in the anti-Fas antibody-treated cultures) (Wilson, D. et al., 1999, Cell Immunol., 194:67). However, parallel cultures of SH-SY5Y neuronal cells did not show any evidence of Fas-induced apoptosis by cell cycle analysis (5.6±3.0% apoptosis in untreated versus 4.6±2.9% in anti-Fas treated cultures). We then treated the cells with Fas Ligand (FasL) constructs, consisting of FasL fused to a linker peptide and a FLAG-tag that facilitate formation of active trimers. These FasL constructs provide physiological ligation of Fas and are highly effective at inducing apoptosis in Jurkat cells (>90% apoptotic cells after 24 hour treatment). However, FasL constructs failed to induce any detectable apoptotic response in the SH-SY5Y cells. Apoptosis induction by FasL constructs in the Jurkat cells was completely abrogated by z-IETD-fink (IETD), a cell permeable peptide which specifically and irreversibly inhibits caspase 8 function (Kataoka, T et al., 2000, Curr. Biol., 10:640; Wosik, K. et al., 2001, Glia, 33:217). Conversely, blockade of the ERK pathway by the MEK1-specific inhibitor PD98059 had no effect on Jurkat apoptosis. Neither IETD nor PD98059 had any effect on apoptosis of the FasL-treated neuroblastoma cells. In addition, we examined replicate populations of cells for evidence of caspase 8 cleavage as an indication of Fas-induced apoptosis, after 4 hours in culture with control antibodies or anti-Fas antibodies.

Again, Jurkat cells demonstrated both spontaneous and anti-Fas-induced caspase 8 breakdown, but caspase 8 cleavage was not induced by anti-Fas antibody treatment in SH-SY5Y cells. Furthermore, SH-SY5Y cells expressed dramatically less caspase 8 than did Jurkat cells, although they expressed similar levels of FLIP and of Fas. Low levels of caspase 8 expression and inefficient (or non-physiological) Fas ligation by anti-Fas antibody could have masked potential Fas-induced caspase 8 cleavage in SH-SY5Y cells. Therefore, we stimulated the cells with FasL constructs and normalized protein loading to equivalent caspase 8 (instead of equivalent total protein). In Jurkat cells, FasL constructs were highly effective at inducing caspase 8 cleavage (caspase 8 cleavage increased by more than 60% in FasL-treated versus untreated cells, whereas anti-Fas antibody was relatively inefficient, producing approximately 20% more caspase 8 cleavage in treated versus untreated cells). Caspase 8 cleavage was reduced to background levels by IETD treatment, and was unaffected by ERK pathway inhibition with PD98059. In contrast, SH-SY5Y neurons showed no increase in caspase 8 cleavage in response to Fas engagement.

These data demonstrate that Fas engagement induced effective, IETD-inhabitable apoptosis and caspase 8 cleavage in Jurkat cells, but failed to activate caspase 8 cleavage and apoptosis in SH-SY5Y neuroblastoma cells.

Fas engagement induces ERIC activation and p35 expression in SY-SH5Y cells. Fas cross-linking has been reported to activate ERIC in glioma cells (Shinohara, H. et al., 2000, Cancer Research, 60:1766). We found that stimulation of SH-SY5Y neuroblastoma cells with anti-Fas antibodies or FasL constructs triggered ERK activation, as evidenced by dual phosphorylation of ERK at threonine 202 and tyrosine 204. Fas-induced ERK activation was inhibited by PD98059, but appeared to be caspase 8 independent as it was not affected by IETD. ERIC activation was detected within five minutes of Fas engagement, and persisted for up to 150 minutes.

Sustained activation of ERK for more than 80 minutes was necessary and sufficient for NGF-inducible neurite outgrowth, and p35 upregulation driven by sustained ERK activation is an essential component of this pathway (Pang, L. et al., 1995, J. Biol. Chem., 270:13585; Harada, T. et al., 2001, Nat. Cell Biol., 3:453). Thus, we examined SH-SY5Y cells for Fas-induced p35 expression. After 24 h incubation with anti-Fas antibodies, p35 was upregulated in SH-SY5Y cells. p35 upregulation was prevented by inhibition of ERIC activation with PD98059, but not with SB202474, a negative control inhibitor that does not affect ERK activation. Thus, Fas engagement induced sustained ERK activation and MEK1/ERK-dependent p35 expression in SH-SY5Y neuroblastoma cells.

Fas engagement induces neurite outgrowth and p35 upregulation in dorsal root ganglia (DRG) explants. To determine whether Fas-induced activation of the ERK/p35 pathway in neuroblastoma cells correlated with neurite sprouting in primary neurons, we used the DRG neurite outgrowth assay, a well characterized model for axon regeneration from primary neurons in vitro (Harada, T. et al., 2001, Nat. Cell Biol., 3:453). DRGs are collections of sensory neuron cell bodies, together with supporting Schwann cells and fibroblasts. Primary DRG explants from neonatal mice sprout neurites in the presence of nerve growth factor (NGF), but do not regenerate neurites in the absence of growth factors, providing a model for neural regeneration driven by external signals. We examined Fas expression selectively on the DRG neurons by flow cytometric analysis of DRG cells positive for neuron-specific tubulin III, thus excluding Schwann cells and other non-neural cell types from the analysis. We found that DRG neurons express Fas. The uniform shift in fluorescence intensity, without the presence of distinct subpopulations, indicates uniform Fas expression on the DRG neurons (geometric mean fluorescence intensity shift from 10.95 with negative control staining to 22.39 with anti-Fas staining). DRG explants were cultured with anti-Fas antibodies, with isotype-matched control antibodies (as a negative control), or with NGF (as a positive control). We found that Fas cross-linking induced rapid, robust neurite outgrowth from the DRGs. Fas-induced neurite outgrowth was indistinguishable from NGF-induced neurite outgrowth kinetically and by morphological criteria (neurite numbers, length, and branching), and could not be differentiated by blinded observers. Fas-induced neurite growth was robust and prolific, completely filling the wells of 96-well plates after two weeks in culture. Fas-stimulated DRGs continued to extend robust neurites and showed no morphological signs of apoptosis even after two weeks in culture. Thus, Fas engagement induced a rapid and prolific neurite outgrowth response in primary sensory neurons.

As in SH-SY5Y neurons, Fas engagement induced p35 upregulation in DRG explants after 24-hour stimulation. Fas engagement and NGF treatment induced a 4.8 and 3.3-fold upregulation, respectively, compared to untreated DRGs, calculated by normalizing p35 to actin on Western blots. Together, these data demonstrate that Fas engagement induces p35 upregulation and neurite outgrowth in primary sensory neurons. Fas-induced neurite growth is independent of NGF and caspase 8 function, but dependent on ERK activation. We studied the mechanism of Fas-induced neurite growth by verifying that we stimulated, and did not block, the Fas receptor. FasL constructs, which are highly effective at inducing apoptosis in Jurkat cells and hence are able to induce potent signals through Fas, also induced robust neurite growth. Furthermore, neutralizing anti-Fas-Ligand antibodies, in contrast to FasL constructs and anti-Fas antibodies, did not induce neurite outgrowth, indicating that disruption of endogenous Fas/Fas Ligand interactions was not sufficient to induce neurite outgrowth.

We then investigated the mechanism of Fas-induced neurite outgrowth by treating the DRGs with inhibitors to potential mediators of Fas activity. As NGF- and Fas-induced neurite growth were indistinguishable morphologically, we reasoned that Fas engagement might be triggering the release of NGF and thus acting indirectly on neurite growth via NGF secretion. However, Fas-induced neurite growth was not blocked by neutralizing anti-NGF antibodies, suggesting that Fas-induced neuritogenesis was independent of NGF secretion.

Fas-induced neurite growth was not affected by treatment with IETD, consistent with caspase 8 independence. Conversely, Fas-induced neurite growth was prevented by treatment with the ERK pathway inhibitor PD98059. Although PD98059 proved to be a potent inhibitor of neurite growth, it did not kill the neurons, since we could wash out the inhibitor after two days in culture, and then restimulate the inhibited DRGs with NGF or FasL constructs and still obtain a healthy neurite growth response. Furthermore, PD98059 was not toxic to other DRG cells such as Schwann cells, as viable, adherent cells could be seen surrounding the DRG despite the absence of neurite growth. Thus, neurite outgrowth was specifically inhibited by suppressing the ERK pathway, despite continued viability and neuritogenic potential of the DRG neurons. Inhibition of the MEK1/ERK pathway blocked neurite outgrowth stimulated by either NGF or anti-Fas antibody, suggesting that Fas and NGF receptor signals converge on a common ERK-dependent pathway.

The Fas pathway leading to neurite growth appears to be completely independent of caspase 8, as it is not blocked by IETD, nor is it compromised in lpr-cg mice (Kimura, M. et al., 1994, Int. Rev. Immunol., 11:193-198). Lpr-cg mice bear a mutation in the death domain of Fas, preventing its coupling to the caspase cascade, but express normal levels of cell surface Fas. In contrast, DRGs from lpr mice, which bear a mutation resulting in reduced Fas expression (Nagata, S. et al., 1995, Science, 267:1449), did not grow neurites in response to anti-Fas antibody, demonstrating the specificity of the response to Fas engagement.

Finally, we confirmed that Fas-induced neurite outgrowth was mediated directly via the neurons, independently of glial cells, by examining dissociated DRG cultures in which glial growth had been suppressed with cytosine arabinoside. Consistent with our results in explant cultures, we found that neurite outgrowth was induced by anti-Fas antibodies or NGF, but was absent if the neurons were left untreated. Together with our finding in neuroblastoma cells, these data demonstrate that Fas ligation on the neuron is sufficient to mediate Fas-induced neurite growth.

Endogenous Fas expression accelerates in vivo functional recovery after sciatic nerve trauma. The final criterion for successful regeneration is functional recovery in vivo. Using a mouse sciatic nerve crush model, the kinetics of nerve regeneration can be followed by walking track analysis, which quantifies recovery of normal gait and the ability to bear weight on the injured limb (De Medinaceli, L. et al., 1982, Exp. Neyrol., 77:634). To examine the contribution of physiological Fas expression to nerve regeneration in vivo, we compared the rate of functional recovery after injury in wild type mice and mice with defective Fas expression (lpr mice) (Nagata, S. et al., 1995, Science, 267:1449). In both strains, walking track analysis revealed a maximal deficit immediately after sciatic nerve crush injury, which progressively resolved in wild-type mice by 20 days post injury. However, Fas-deficient lpr mice experienced significantly delayed recovery after sciatic crush injury, and failed to recover fully by 20 days post-injury. These findings suggest that endogenous Fas expression accelerates peripheral nerve regeneration after crush injury.

To determine whether the regenerative effects of Fas were mediated through the apoptotic pathway, nerve crush studies were conducted in lpr-cg mice, which express normal levels of Fas but bear a point mutation in the death domain of Fas. FasLpr-cg cannot recruit FADD, and thus is unable to trigger Fas-induced apoptosis (Kimura, M. et al., 1994, Int. Rev. Immunol., 11:193-198). However, FasLpr-cg can mediate Fas proliferative effects (Desbarats, J. et al., 1999, PNAS, 96:8104; Desbarats, J. et al., 2000, Nat. Med., 6:920), and lpr-cg. DRGs (unlike lpr DRGs) extend neurites in response to Fas engagement. We found that in lpr-cg mice, unlike in lpr mice, functional recovery was not delayed, and in fact appeared slightly, though not significantly, accelerated. Thus, Fas-induced apoptotic signals do not significantly affect the rate of recovery, while Fas-induced growth signals significantly accelerate recovery.

We examined the effect of in vivo administration of anti Fas antibody at the nerve crush site. We found that functional recovery was significantly accelerated in mice treated with anti-Fas antibodies injected into the nerve at the time of crush injury, compared with mice treated with isotype-matched control antibodies. Histological recovery was also improved by anti-Fas antibody. An increased number of myelinated profiles, more uniform axon diameters, more numerous Schwann cell nuclei, and less vacuolation and inflammatory infiltrate was evident in anti-Fas-treated sciatic nerve, compared with control antibody-treated sciatic nerve, eight days after injury. Thus, endogenous Fas expression likely contributes to peripheral nerve regeneration in vivo, and recovery was further accelerated by the administration of anti-Fas antibodies at the site of the crush injury.

Thus, we have shown that Fas engagement on peripheral neurons stimulates axon regeneration; that decreased endogenous Fas expression can delay nerve regeneration, that exogenous administration of anti-Fas antibody accelerates nerve regeneration; and that Fas can activate the ERK signaling pathway in CNS neurons.

### Example 2

### Oxidative Stress Promotes Increases in Immune Recognition Molecules on Neurons

We have found that C17.2 cells express Fas (CD95) and B7.1 co-stimulatory molecule and that the levels of their expression on the cell surface increase following 24-hr exposure to subcytotoxic concentrations of H₂O₂, 0.25 mM. We have measured the levels of intracellular H₂O₂ in these cells. We have found that UCP-2 is expressed by C17.2 cells and that it increases with passage number. C172 cells from passage 15 contained 4-fold higher amount of UCP-2 than C17.2 cells at passage 11.

Ts65Dn mice and controls fed regular and fatty acid enriched diet. The effects of dietary supplementation with alpha lipoic acid (LA) and N-acetylcarnitine (ALCAR) on the Ts65Dn mouse and the strain matched control animals have been examined. This combination of fatty acids has been shown to ameliorate cognitive loss with aging in rats, and may do the same for beagle dogs. We attempted this supplementation with old (18 months) Ts65Dn mice. Our results demonstrate that supplementation makes the Ts65Dn behavior on the Morris Water Maze much worse, which is a completely unexpected result. The dietary supplementation shows a trend toward lowering of oxidative stress in the normal mice. The Ts65Dn mice have elevated levels of oxidative stress without supplementation, and supplementation trends toward increasing rather than decreasing oxidative stress in these mice.

We measured mitochondrial membrane potential, levels of reactive oxygen, and cell surface expression of B7.1, a costimulatory molecule important in T cell activation. Our results indicate that normal mice effectively had reduced levels of reactive intermediates, but in sharp contrast, the effect of the diet on the Ts6Dn mice was the reverse and the levels of reactive intermediates increased as a result of the diet. The data is presented in the tables below. Thus, a subject with DS may react differently to these widely used dietary supplements than a person without DS.

Engagement of Fas expressed on the cell surface of neurons can act as a survival and/or regenerative signal, and dysfunctional or absent Fas signals that result from an altered metabolic state in Down Syndrome may lead to neuronal degeneration.

Trisomy Mouse Model SPLEENS
On Special Diet vs. Not on Special Diet
DCFDA, Mito Tracker, & B7.1 in Triplicate
All On Diet

| | DCFDA #1 | DCFDA #2 | DCFDA #3 | Mean | Stdev |
|---|---|---|---|---|---|
| Mouse #13 | 1071 | 1092 | 1156 | 1106.333333 | 44.2756517 |
| Mouse #14 | 658 | 564 | 601 | 607.6666667 | 47.35328218 |
| Mouse #15 | 442 | | 185 | 313.5 | 181.7264428 |
| | B7.1 #1 | B7.1 #2 | B7.1 #3 | Mean | Stdev |
| Mouse #13 | 1.45 | 1.68 | 1.64 | 1.59 | 0.122882057 |
| Mouse #14 | 1.63 | 1.68 | 1.68 | 1.663333333 | 0.028867513 |
| Mouse #15 | 1.27 | 1.24 | 1.25 | 1.253333333 | 0.015275252 |
| M2 Peaks Only | Mito #1 | Mito #2 | Mito #3 | Mean | Stdev |
| Mouse #13 | 34.7 | 32.9 | 25.1 | 30.9 | 5.102940329 |
| Mouse #14 | 28.1 | 25.8 | 25.9 | 27.5 | 1.4 |
| Mouse #15 | 22.4 | 20.1 | 20.8 | 21.1 | 1.178982612 |

Trisomy Mouse Model SPLEENS
On Special Diet vs. Not on Special Diet
DCFDA, Mito Tracker, & B7.1 In Triplicate
No Special Diet

| | DCFDA #1 | DCFDA #2 | DCFDA #3 | Mean | Stdev | |
|---|---|---|---|---|---|---|
| Mouse #26 | 477 | 445 | 470 | 464 | 16.82260384 | *M2 |
| Mouse #28 | 668 | 593 | 601 | 620.6666667 | 41.18656739 | *All |
| Mouse #29 | 450 | 471 | 472 | 464.3333333 | 12.42309677 | *M2 |
| | | | | | | *Maj. |
| Mouse #30 | 874 | 839 | 801 | 838 | 36.51027253 | Of Pop |
| | B7.1 #1 | B7.1 #2 | B7.1 #3 | Mean | Stdev | |
| Mouse #26 | 1.5 | 1.5 | 1.5 | 1.5 | 0 | |
| Mouse #28 | 17.64 | 17.23 | 17.99 | 17.62 | 0.380394532 | *M1 |
| Mouse #29 | 1.61 | 1.65 | 1.63 | 1.63 | 0.02 | |
| Mouse #30 | 1.45 | 1.46 | 1.4 | 1.436666667 | 0.032145503 | |
| | Mito #1 | Mito #2 | Mito #3 | Mean | Stdev | |
| Mouse #26 | 13 | 13 | 12 | 12.66666667 | 0.577350269 | *All |
| Mouse #28 | 23.4 | 83.2 | 9.5 | 38.7 | 39.15980082 | *M2 |
| Mouse #29 | 30 | 20 | 22 | 24 | 5.291502622 | *M2 |
| Mouse #30 | 30 | 30.5 | 31.5 | 30.66666667 | 0.763762616 | *M2 |

| | | |
|---|---|---|
| Control | Control | Control |
| #1 | #2 | #3 |

| Mean | StDev | B-Cells | B-Cells | B-Cells | Mean | StDev | | |
|---|---|---|---|---|---|---|---|---|
| 35.94667 | 0.977002 | 134 | 133 | 124 | 130.3333 | 5.507571 | 0.977002 | 5.01417 |
| 147 | 35.79106 | 371 | 359 | 481 | 403.6667 | 67.24086 | 35.79106 | 43.00388 |
| 312 | 39.15354 | 2020 | 1924 | 2136 | 2026.667 | 106.1571 | 39.15353 | 151.0177 |
| 0.953333 | 0.085049 | 7.18 | 10.87 | 7 | 8.35 | 2.184239 | 0.085049 | 0.140119 |
| 2.29 | 0.072111 | 83.16 | 81.5 | 80.91 | 81.85667 | 1.166633 | 0.072111 | 1.05633 |
| 3.64 | 0.247588 | 117 | 140 | 116 | 124.3333 | 13.57694 | 0.247588 | 2.997354 |
| | | TS #1 | TS #2 | TS #3 | | | | |
| Mean | StDev | B-Cells | B-Cells | B-Cells | Mean | StDev | | |
| 30.35 | 5.01417 | 150 | 91.04 | 145 | 128.68 | 32.69292 | 5.507571 | 32.69292 |
| 172.6667 | 43.00388 | 480 | 543 | 407 | 476.6667 | 68.06125 | 67.24086 | 68.06125 |
| 310.6667 | 151.0177 | 2185 | 1111 | 2636 | 1977.333 | 783.4222 | 106.1571 | 783.4222 |
| 0.963333 | 0.140119 | 9.64 | 12.51 | 7.31 | 9.82 | 2.604669 | 2.184239 | 2.604669 |
| 2.573333 | 1.05633 | 251 | 67.64 | | 159.32 | 129.6551 | 1.166633 | |
| 4.046667 | 2.997354 | 660 | 117 | | 388.5 | 383.959 | 13.57694 | |

Trisomy Mouse Model SPLEENS
On Special Diet vs. Not on Special Diet
DCFDA, Mito Tracker, & B7.1 In Triplicate
All On Diet

| | DCFDA #1 | DCFDA #2 | DCFDA #3 | Mean | Stdev |
|---|---|---|---|---|---|
| Mouse #13 | 1071 | 1092 | 1156 | 1106.333333 | 44.2756517 |
| Mouse #14 | 658 | 564 | 601 | 607.6666667 | 47.35328218 |
| Mouse #15 | 442 | | 185 | 313.5 | 181.7264428 |
| | B7.1 #1 | B7.1 #2 | B7.1 #3 | Mean | Stdev |
| Mouse #13 | 1.45 | 1.68 | 1.64 | 1.59 | 0.122882057 |
| Mouse #14 | 1.63 | 1.68 | 1.68 | 1.663333333 | 0.028867513 |
| Mouse #15 | 1.27 | 1.24 | 1.25 | 1.253333333 | 0.015275252 |
| M2 Peaks Only | Mito #1 | Mito #2 | Mito #3 | Mean | Stdev |
| Mouse #13 | 34.7 | 32.9 | 25.1 | 30.9 | 5.102940329 |
| Mouse #14 | 28.1 | 28.5 | 25.9 | 27.5 | 1.4 |
| Mouse #15 | 22.4 | 20.1 | 20.8 | 21.1 | 1.178982612 |

Trisomy Mouse Model SPLEENS
On Special Diet vs. Not on Special Diet
DCFDA, Mito Tracker, & B7.1 In Triplicate
No Special Diet

| | DCFDA #1 | DCFDA #2 | DCFDA #3 | Mean | Stdev | |
|---|---|---|---|---|---|---|
| Mouse #26 | 477 | 445 | 470 | 464 | 16.82260384 | *M2 |
| Mouse #28 | 668 | 593 | 601 | 620.6666667 | 41.18656739 | *All |
| Mouse #29 | 450 | 471 | 472 | 464.3333333 | 12.42309677 | *M2 |
| | | | | | | *Maj. |
| Mouse #30 | 874 | 839 | 801 | 838 | 36.51027253 | Of Pop |
| | B7.1 #1 | B7.1 #2 | B7.1 #3 | Mean | Stdev | |
| Mouse #26 | 1.5 | 1.5 | 1.5 | 1.5 | 0 | |
| Mouse #28 | 17.64 | 17.23 | 17.99 | 17.62 | 0.380394532 | *M1 |
| Mouse #29 | 1.61 | 1.65 | 1.63 | 1.63 | 0.02 | |
| Mouse #30 | 1.45 | 1.46 | 1.4 | 1.436666667 | 0.032145503 | |
| | Mito #1 | Mito #2 | Mito #3 | Mean | Stdev | |
| Mouse #26 | 13 | 13 | 12 | 12.66666667 | 0.577350269 | *All |
| Mouse #28 | 23.4 | 83.2 | 9.5 | 38.7 | 39.15980082 | *M2 |
| Mouse #29 | 30 | 20 | 22 | 24 | 5.291502622 | *M2 |
| Mouse #30 | 30 | 30.5 | 31.5 | 30.66666667 | 0.763762616 | *M2 |

TG Mice vs. Control
Special Diet vs. No Diet

| | B7.1 | DCFDA | | |
|---|---|---|---|---|
| TG Mice On Diet | 1.1 | 2.09 | | |
| TG Mice No Diet | 1.14 | 1.91 | | |
| Control Mice | 1.05 | 1.36 | | |
| On Diet | | | | |
| Control Mice | 1.12 | 1.71 | | |
| No Diet | | | | |
| StDev | 0 | 0.011547 | 0.075498 | 0.030551 |
| StDev | 0 | 0.361709 | 0.09609 | 0.195533 |

TG Mice vs. Control
On Special Diet

| | | | Average | Average | Standard | Standard |
|---|---|---|---|---|---|---|
| | B7.1 | DCFDA | B7.1 | DCFDA | Dev. B7.1 | Dev. DCFDA |
| TG Mouse #1 | 1.68 | 9.4 | 1.1 | 2.09 | | |
| TG Mouse #3 | 1.12 | 2.32 | | | | |
| TG Mouse #6 | 1.08 | 1.86 | | | | |
| Control Mouse #2 | 1.12 | 1.26 | 1.05 | 1.36 | | |
| Control Mouse #4 | 1.06 | 1.45 | | | | |
| Control Mouse #5 | 0.97 | 1.38 | | | | |

TG Mice vs. Control
No Diet
18 Mar. 2002

| | | | Average | Average | Standard | Standard |
|---|---|---|---|---|---|---|
| | B7.1 | DCFDA | B7.1 | DCFDA | Dev. B7.1 | Dev. DCFDA |
| TG Mouse #16 | 1.15 | 2.15 | 1.14 | 1.91 | | |
| TG Mouse #17 | 1.15 | 2.1 | | | | |
| TG Mouse #19 | 1.13 | 1.5 | | | | |
| Control Mouse #18 | 1.25 | 1.7 | 1.12 | 1.71 | | |
| Control Mouse #20 | 1.21 | 1.53 | | | | |
| Control Mouse #21 | 1.19 | 1.92 | | | | |

### Example 3

### Analysis to Determine how Trisomy of Genes on Chromosome 21 Alter Mitochondrial Metabolism, the Levels of Intracellular Reactive Intermediates in Neuron, and Changes in Cell Surface Expression of Fas (CD95)

3. a Characterization of Fas and Fas Ligand Expression on Normal and DS Neurons

The cell surface expression of Fas and Fas Ligand on the H1b and HTk cell lines (Cardenas, A. et al., 2002, Exp. Neurol., 177:159) is assessed. Cell surface Fas is detectable by flow cytometry. Fas Ligand is detected by Western blot analysis. Neurons from the TsDn mice in primary cultures of embryonic mouse cells are also examined for Fas expression. The neurons are double labeled with fluorochrome-conjugated anti-synaptophysin and anti-Fas antibodies. The labeled cells are examined flow cytometrically and by confocal microscopy. If the freshly isolated cholinergic neurons do not spontaneously express Fas, are cultured in increased glucose concentrations, or in the presence of growth factors (such as glial derived neurotrophic factor). These manipulations have resulted in increased Fas expression in other cell types (Newell, M. et al., 1999, Ann. N.Y. Acad. Sci., 887:77). Cells that are positively stained for cell surface Fas are selected.

3.b. The data described above indicate that both glucose-free and glucose-supplemented (5× normal) cell cultures exhibit increased cell surface Fas expression. Fas may be functioning as a sensor of metabolite availability, with any change resulting in upregulated expression so that Fas-mediated signals can subsequently be used to determine cell fate (death versus proliferation).

Glucose-free tissue culture medium (complete RPMI containing fetal bovine serum, glutamine, 2-ME, Hepes, and antibiotics) is used to prepare media containing 0 (glucose-free), 2 (physiological), 5, 10 and 20 g/L glucose, at pH 72. Neuronal cell lines, H1b and HTk cells are cultured for varying times, from 24 to 72 hours, in these media containing increasing concentrations of glucose. The cells are harvested, counted as a measure of proliferative rate, stained with fluorochrome-labeled anti-Fas and anti-FasL antibodies or with fluorochrome labeled isotype control antibodies (PharMingen), and analyzed by multi-parameter flow cytometry. Net Fas and FasL expression is quantitated by subtracting the geometric mean fluorescence of the isotype control-labeled cells from the geometric mean fluorescence of the Fas- or FasL-labeled cells. Flow cytometry is a very sensitive technique and can reproducibly reveal small changes in cell surface expression.

3.c. Does Inhibition of or Defects in, Glucose Utilization Result in Susceptibility to Fas-Induced Death? DS Dependent Defects in Glucose Utilization Results in Fas-Induced Death when Fas is Engaged.

In the absence of glucose, the data show that proliferation is inhibited, Fas expression is increased, and cell viability is decreased in cells which express FasL. Taken together, these observations suggest that Fas may induce apoptosis in glucose-deprived cells. Therefore, defects in glucose utilization in DS results in increases in apoptosis of neurons is tested.

A system of antibodies coated onto plastic tissue culture wells is used to provide a cross-linking stimulus for cell surface receptors. Anti-Fas antibodies (Jo-2, PharMingen) or isotype control antibodies (which do not specifically bind anything on the cell surface, but serve as a control) are coated onto tissue culture wells into which aliquots of H1b or HTK cells or primary mouse neurons from Ts65Dn or strain-matched control animals are plated. The cells are cultured at physiological glucose concentration, in glucose-free medium, or in medium containing 2-deoxy-glucose, an inhibitor of glycolysis. At varying time points during the culture, the cells are harvested, stained with Bauer's DNA-labeling solution, and analyzed by flow cytometry. This staining method allows flow cytometric cell cycle analysis and reveals the percentage of apoptotic, actively cycling, or resting cells. The cells are labeled with 3H-thymidine 16-18 hrs prior to harvest, to quantitate cell proliferation by total DNA synthesis (Desbarats, J et al., 2000, Nat. Med., 6:920). Any difference in apoptosis or cell proliferation between the cells cultured with anti-Fas antibodies compared with those cultured with control antibodies reveals the effects of exogenous Fas engagement under each condition. Similarly, differences between the Ts65Dn and strain-matched controls indicates potential differences in Fas-mediated growth or death.

3.d Metabolic Dysfunction in Ds Resulting from Defects in Mitochondrial Activity.

The mitochondrial respiration system is an important source of intracellular reactive oxygen species and other free radicals. Several groups have shown that the levels of ROI are increased in the DS neurons and that reduced mitochondrial redox state and membrane potential reflect impaired mitochondrial function. Mutations in mtDNA could result in increases in free radicals and reduced ATP levels and together suggest that mitochondrial dysfunction may affect neuronal development and the pathogenesis of DS. Therefore, mitochondrial function in model cell lines and primary cultures of cholinergic neurons will be analyzed.

Mitochondrial Membrane Potential. The data indicate that there may be differences in mitochondrial activity between cells from the Ts65Dn neurons and neurons from strain matched controls. The baseline levels of mitochondrial membrane potential of the model cell lines and primary cells are established. The mitochondrial membrane potential is measured flow cytometrically by incubating cells for 20 minutes at room temperature with 5 mg/ml JC-1 or Mitotracker red fluoresces as a function of increasing mitochondrial membrane potential. The aggregation state and consequently the fluorescence emission of JC-1 changes as the mitochondrial membrane potential is altered. Valinomycin, which collapses the mitochondrial membrane potential is used as a positive control treatment. Flow cytometry permits the examination of up to four fluorescent markers concurrently; thus, the cells are counter stained with anti-Fas antibodies. The flow cytometric data is confirmed by using dual simultaneous measurements of oxygen consumption using electrical probes and membrane potential as confirmation (Brand, M et al., 1993, Biochem. J., 291:739).

Comparative measurements of rate of glucose utilization by quantitating conversion of _H-glucose to _H—H₂O. Glucose utilization is measured by the method of Ashcroft (Ashcroft, S. et al., 1972, Biochem. J., 126:525). Briefly, cells are incubated in 100 microliters of appropriate medium, glucose (2.8-27.7 mM) 2 microCi D-³H-glucose. The reaction is carried out in a 1 ml cup in a rubber stoppered scintillation vial with 500 microliters of distilled water surrounding the cup. Glucose metabolism is stopped with 100 microliters of a 1 mol/l HCl injected through the stopper into the cup. The samples are incubated overnight thereafter at 37° C. to allow equilibration of the ³H—H₂O in the reaction cup and the distilled water, the ³H—H₂O in the reaction cup and the distilled water is quantitated using a liquid scintillation counter. This technique allows a determination of the rate of glucose uptake as an indication of glycolysis.

Measurements of rate of glucose oxidation by uptake and metabolism of ¹⁴C-glucose and conversion to CO₂. Glucose oxidation is measured by incubating cells for 90 min at 37° C. in 100 microliters of reaction buffer, glucose (2.8, 8.3, 27.7 mmol/l), 1.7 mCi (U-¹⁴C glucose. The reaction is carried out in a 1 ml cup in a 20 ml scintillation vial capped by a rubber stopper with a center well that contains filter paper. Metabolism is stopped and CO₂ liberated with 300 microliters of a 1 mol/l HCl injected through the stopper into the cup containing the cells. CO₂ is trapped in the filter paper by injecting 10 ml 1 mol/l KOH into the center well, followed 2 hours later by liquid scintillation counting. Tubes containing NaHCO₃ and no cells are used to estimate the recovery of ¹⁴CO₂ in the filter paper which should be routinely close to 100%. This technique provides information as to rate of respiration.

Fatty Acid Oxidation. Fatty acid metabolism in Down Syndrome has been well studied and is known to be abnormal giving rise to increased rates of atherogenesis and diabetes. Rates of oleate consumption are measured by incubating cells for 90 min at 37° C. in 100 microliters of reaction buffer, oleic acid, and increasing concentrations of glucose (2.8, 8.3, 27.7 mmol/l), 1.7 mCi (U-¹⁴C oleic acid), and cold oleate. The reaction is carried out in a 1 ml cup in a 20 ml scintillation vial capped by a rubber stopper with a center well that contains filter paper. Metabolism is stopped and CO₂ liberated with 300 microliters of 1 mol/l HCl injected through the stopper into the cup containing the cells. CO₂ is trapped in the filter paper by injecting 10 ml 1 mol/l KOH into the center well, followed 2 hours later by liquid scintillation counting. Tubes containing NaHCO₃ and no cells are used to estimate the recovery of ¹⁴CO₂ in the filter paper, routinely close to 100% (Ashcroft, S. et al., 1972, Biochem. J., 126:525).

Flow Cytometric Measurements of Reactive Oxygen intermediates. Cells are stained for intracellular H₂O₂ using 6-carboxy-2′,7′-dichlorodihydrofluorescein diacetate (DCF-DA, Molecular Probes, Eugene, Oreg.). Briefly, cells are incubated with 1 mM DCF-DA for 20 minutes, washed twice in PBS containing 5% fetal calf serum and analyzed flow cytometrically.

3. e. Transfection of UCP to prevent or protect from reactive intermediates. The mitochondrial dysfunction in Down Syndrome results in overproduction of free radicals and/or failure to be protected from the free radicals once formed. The damage from oxidative stress in Down Syndrome resulting from impaired protective strategies and failure to burn fat is assessed. Overexpression of UCP2 on the H1T and H2b cell line is tested and levels of intracellular free radicals measured flow cytometrically.

For tightly controlled expression of UCP, the Tet-On expression system in combination with the tetracycline-controlled transcriptional silencer (tTS) is used (Freundlieb, M. et al., 1999, J. Gene Med., 1:4). The UCP-2 is co-expressed with enhanced green fluorescent protein (EGFP) from a bidirectional tetracycline responsive promoter (Gossen, M. et al., 1995, Science, 268:1766). Cells that show tight regulation of UCP-2 expression by measuring fluorescence from EGFP are selected. Stable cell lines (Tet-On cell lines) that express the reverse tetracycline-responsive transcription activator (rtTA) and the tetracycline-controlled transcriptional silencer (tTS) (Gossen, M. et al., 1995, Science, 268:1766; Freundlieb, M. et al., 1999, J. Gene. Med., 1:4) are generated. Co-transfection of H1T and H2b cells with a pTet-On (Clontech) derivative and pTet-tTS (Clontech) and selection of stable transfectants is accomplished. The neomycin resistance gene of pTet-On has been replaced with the puromycin resistance gene from pKO SelectPuro (Stratagene) in the C17.2 neural stem cell lines. Plasmids: pTet-On, pTet-tTS, pBI-EGFP, and pTK-Hyg are purchased from Clontech and pKO SelectPuro is obtained from Stratagene. The neomycin resistance gene of pTet-On is cut out by XhoI digestion. The puromycin resistance gene of pKO SelectPuro is isolated by digestion with AscI and inserted into the XhoI site of pTet-On using the XhoI linker. The DNA coding the F-HA-UCP-2 is inserted into pBI-EGFP digested with PvuII and MluI using the MluI linker. The transfection is carried out using the calcium phosphate method. In the second phase, the coding sequence of the mouse UCP-2 (e.g. ATCC accession number NM_{—}011671) under control of the tetracycline-response element (TRE) will be introduced into the Tet-On cell clone derived from the H1b and Htk neuronal cell lines. In order to detect the UCP-2 in later studies a FLAG and hemagglutinin (HA) epitope tagged version of UCP-2 is employed. The coding sequence of the HA-UCP-2 is subcloned into pBI-EGFP (Clontech), and used to co-express the genes of interest and EGFP from a bidirectional tetracycline-responsive promoter. pBI-EGFP containing the F-HA-UCP-2 and pTK-Hyg (Clontech) is co-transfected into the Tet-On cells derived from H1T and H2b cells using the calcium phosphate method. S table transfectants are selected and cloned in the presence of hygromycin. Clones that show the least amount of EGFP expression in the absence of doxycycline and the highest EGFP expression in the presence of doxycycline are selected. To determine if oxidative stress occurs to a lesser degree in HTk.UCP-2 cells than in control cells, the intracellular levels of H₂O₂ in the cell lines before and after exogenous stressors staining of the NSCs is analyzed. Stained cells are analyzed for the intracellular H₂O₂ and cell death (PI staining) using flow cytometry. The degree of cell death is examined using TUNEL method that detects DNA cleavage.

### Example 4

### Determination of the Signaling Pathways Triggered by Fas Engagement on Neurons from Down Syndrome Model Mice

Established, characterized cell lines and cultures of mouse neurons, derived from hippocampus of normal and Ts65Dn mice, are used as in vitro models of hippocampal, cholinergic neurons (Cardenas, A. et al., 2002, Exp. Neurol., 177:159). Whether the effects of anti-Fas treatment can be blocked by inhibition of the caspase or ERK cascades is determined.

The Fas death pathway has been extensively studied, as detailed above. The MEK/ERK signaling cascade is the only pathway so far implicated in Fas-mediated growth, and has recently been studied in dopaminergic neurons. The activation of the ERK pathway by Western blotting with antibodies specific for double phosphorylated at threonine 202 and tyrosine 204 on the ERK molecule is examined. Whether the MEK/ERK pathway can account for all the stimulatory effects (either proliferation or differentiation) of Fas engagement is determined by treating the neurons with the selective MEK inhibitors. Whether blocking the ERK pathway facilitates the generation of apoptotic signals through Fas is determined. Activation of the caspase cascade is measured by flow cytometry of cells loaded with fluorogenic caspase substrates. We will use IETD, a specific blocker of caspase 8 (FLICE, which associates with FADD), and the global caspase inhibitor z-VAD.

In summary, whether Fas apoptotic and stimulatory effects are the same or different between cells of normal and mouse models of trisomy, including Ts65Dn, and cell lines from Ts16 is determined. Whether apoptotic and stimulatory effects can be interconverted by treating the cells alternatively with either the MEK/ERK inhibitors or with the specific caspase 8 inhibitor IETD is determined.

Recently, ERK activation has been shown to induce p35, a neuron specific activator of cyclin-dependent kinase 5, which in turn mediates neurite outgrowth (Harada, T. et al., 2001, Nat. Cell Biol., 3:453). Thus, p35 is likely a downstream effector for Fas-induced neurite outgrowth. The induction of p35 is evaluated by Western blot analysis of Fas stimulated cell lines and primary neural cell cultures.

### Example 5

### Role of Fas in Regulating Neural Generation

### Determination of Whether Alterations in Metabolism Render the Neurons More or Less Susceptible to Fas-Induced Death and Whether Fas Engagement, Under the Appropriate Metabolic Conditions, Stimulates Neuron Recovery.

Alterations in metabolic activity in Downs Syndrome results in dysregulated cell death in the developing brain is tested. Extensive analysis on the susceptibility of neurons from Down Syndrome model mice to Fas-induced apoptosis is performed. Well characterized modulators of mitochondrial oxidative stress in cell lines, and immediately ex vivo neurons derived from Down Syndrome model mice are used to test susceptibility of the cells to Fas-induced death. Cells overexpressing the mitochondrial uncoupling proteins are used to determine if efficient mitochondrial uncoupling and fatty acid oxidation prevents Fas-induced death in UCP2 transfected H1b and HTk cell lines.

5. a. Effects of Fas engagement on cell fate. Fas engagement can induce apoptosis, proliferation, or differentiation (Desbarats, J. et al., 1999, PNAS, 96:8104; Desbarats, J. et al., Nat. Med., 6:920). The H1b and HTk cell lines are examined for apoptosis by flow cytomtric cell cycle analysis, which reveals nuclei with a <2N DNA (Desbarats, J. et al., 1999, PNAS, 96:8104). Proliferation is quantified by tritiated thymidine incorporation and cell counting. Differentiation is detected by microscopic examination for neurite elongation and branching. These parameters are examined in primary cholinergic neurons by immunohistochemical labeling for choline acetyltransferase, synaptophysin, and enolase. Other cell types including T cells, lymphocytes, hepatocytes, and dopaminergic neurons, have been used to convert Fas-induced apoptosis to Fas-mediated proliferation by manipulating the cell and its metabolism and environment (Desbarats, J. et al., 1999, PNAS, 96:8104; Desbarats, J. et al., Nat. Med., 6:920).

5. b. Uncoupling proteins. To assess whether protection from reactive oxygen intermediates promotes survival of neurons from DS mitochondrial uncoupling proteins UCP-2 (Fleury, C. et al., 1997, Nature, 15:269) and brain specific UCP-4 (Sanchis, D. et al., 1999, J. Biol. Chem., Science, 268:1766) H1T and HTk cells are stably overexpressed in a regulatable fashion to generate H1b.UCP-2, HTk.UCP-2, H1B. UCP-4 and HTk.UCP-4 cell lines. UCP decreases reactive oxygen species inside mitochondria. Expression of ubiquitous UCP-2 or brain-specific UCP-4 may reduce the effect of trisomy on DS neuron oxidative stress. Initially, stable tetracycline inducible (Tet-On) cell lines are generated by co-transfection of H1b cells with a tetracycline-responsive transcription activator (rtTA) and tetracycline-controlled transcriptional silencer (ITS). UCP expressing DS cell lines are generated by transfecting Tet-On H1b and HTk cells with a construct containing the UCP-2 or -4 genes under control of the tetracycline-response element (TRE). Stable transfectants of the cells are clonally selected, expanded and used. Subsequently, the effects of overexpression of UCPs on cell surface Fas expression and susceptibility to Fas-induced death is measured.

5. c. Crossing Ts65Dn mice with lpr, lpr.cg, and gld mutant mice. Defects in Fas-mediated death or Fas-induced proliferation on the metabolic state in Ts65Dn mice are determined by cross breeding Ts65Dn with 65DC3H.lpr, C3H.gld, and C3H.gld mice (Sakic, B. et al., 2002, J. Neuroimmunol., 129:84). If the defect in Ts65Dn is too much cell death, the lpr mutation could store gain of function. Alternatively, if the defect in metabolism in TsDn prevents regulated cell death, the lpr mutation could worsen the condition. The lpr.cg mutation results in deficient Fas-induced death, but the ability to promote Fas-dependent proliferation is intact. Crossing the Ts65Dn with the lpr.cg will determine if the neuronal dysfunction results from failure to die or if it involves both Fas-induced death and accelerated growth. To distinguish between Fas or its ligand, Fas Ligand, being defective in DS, Ts65Dn are cross bred with C3H.gld animals.

### Example 6

### Presence of UCP in Neuronal Stem Cells

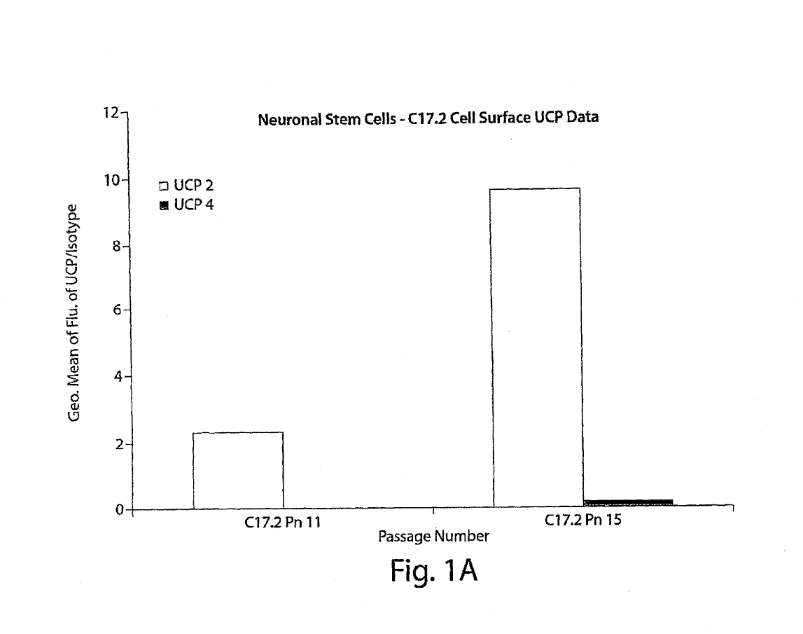

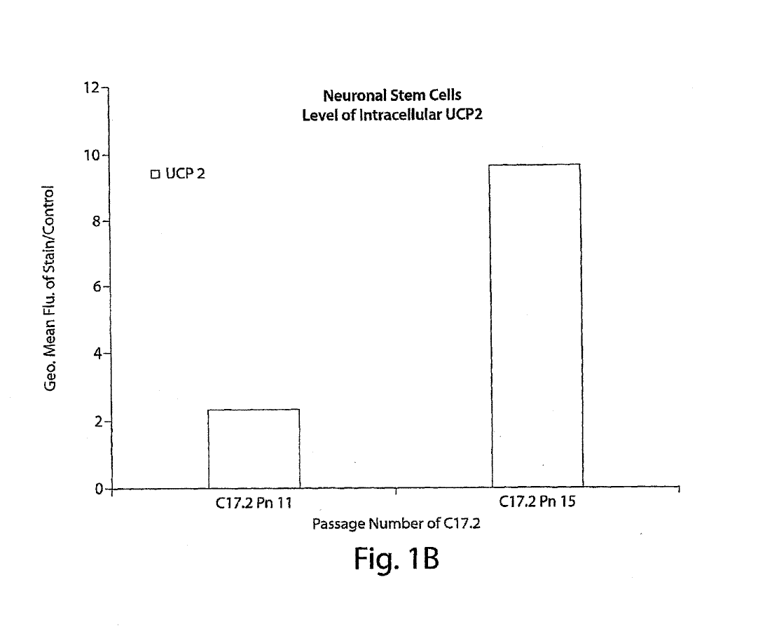

In these experiments, C17.2 mouse neuronal stem cells (a kind gift from Dr. Evan Schnyder, Harvard) were cultured as described in Methods above. However, we harvested cells at various passages as a function of time after thaw from cryostorage. The cells were harvested and stained with either Anti-UCP2 antibody or Anti-UCP4 (Alpha Diagnostics) as indicated. Intact cells or cells that had been permeabilized were stained to determine relative amounts of signal inside versus the cell surface of the stem cells, as indicated. The results are shown in the graphs of FIG. 1a (cell surface UCP) and 1b (intracellular UCP).

### Example 7

### Neuronal Stem Cells Respond to H₂O₂ with Increased B7 and Fas

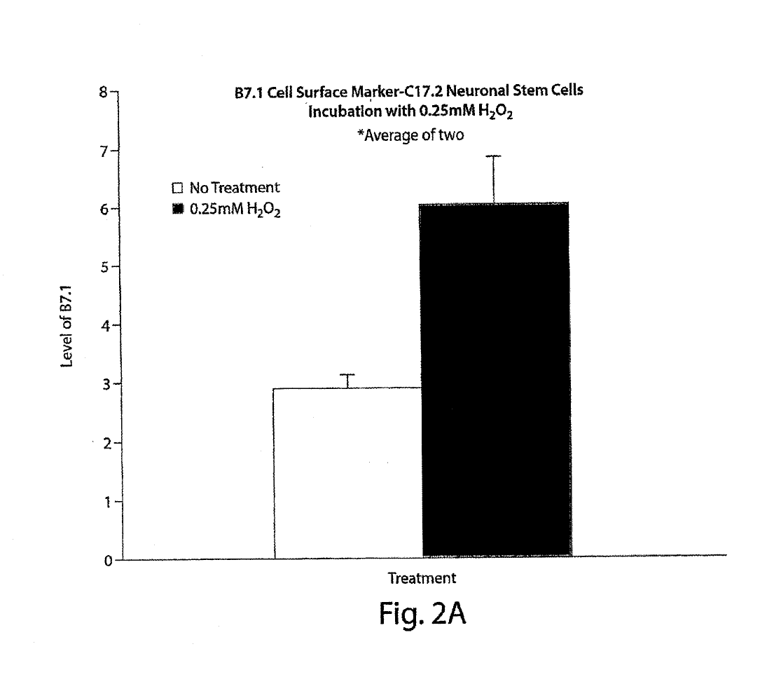

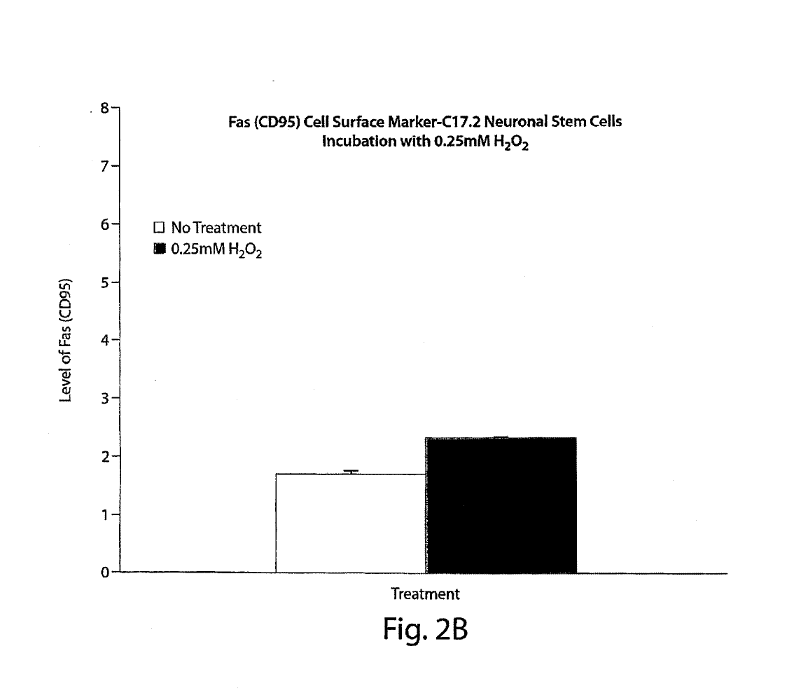

C17.2 mouse neuronal stem cells (a kind gift from Dr. Evan Schnyder, Harvard) were cultured as described in the Methods above. The cells were treated or not with H₂O₂ at the concentration indicated on the graphs of FIG. 2. The cells were harvested and stained with Anti-B71 (FIG. 2a) or Anti-Fas (CD95) (FIG. 2b) antibodies (Pharmingen) as indicated.

### Example 8

### Assessment of Cell Death in Mouse Oligodendrocyte Cells in Response to H₂O₂

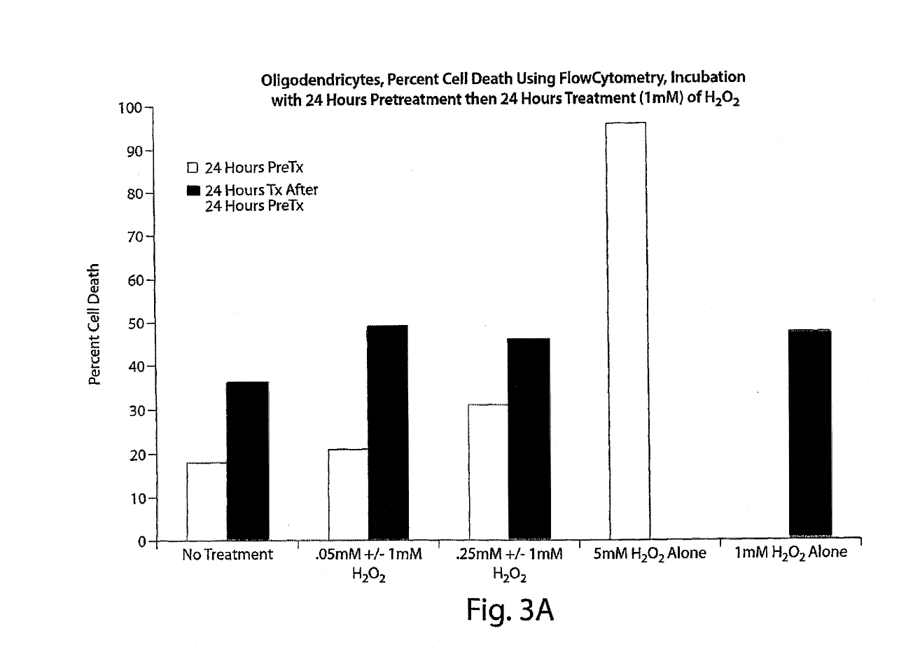

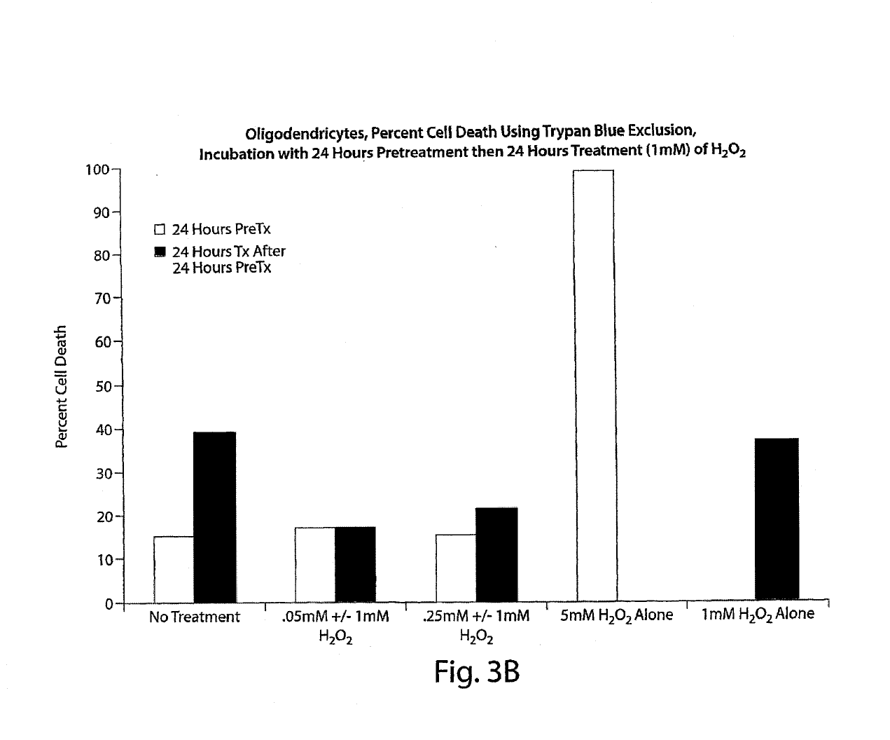

Mouse oligodendrocyte cells (a kind gift from Dr. Adrian Cameron, University of Kentucky) were cultured as described in Methods. The cells were pre-treated or not with H₂O₂ at the concentration indicated on the graphs of FIG. 3. Following pre-treatment the cells were incubated with a higher concentration of H₂O₂ for an additional time frame (indicated on the graphs). The cells were harvested and analyzed for percent death both flow cytometrically (FIG. 3a) as described in the Methods and by Trypan Blue Exclusion (FIG. 3b).

### Example 9

### Assessment of Cell Death, Cell Surface Fas, and Mitotracker Fluorescence in Mouse Oligodendrocyte Cells in Response to H₂O₂

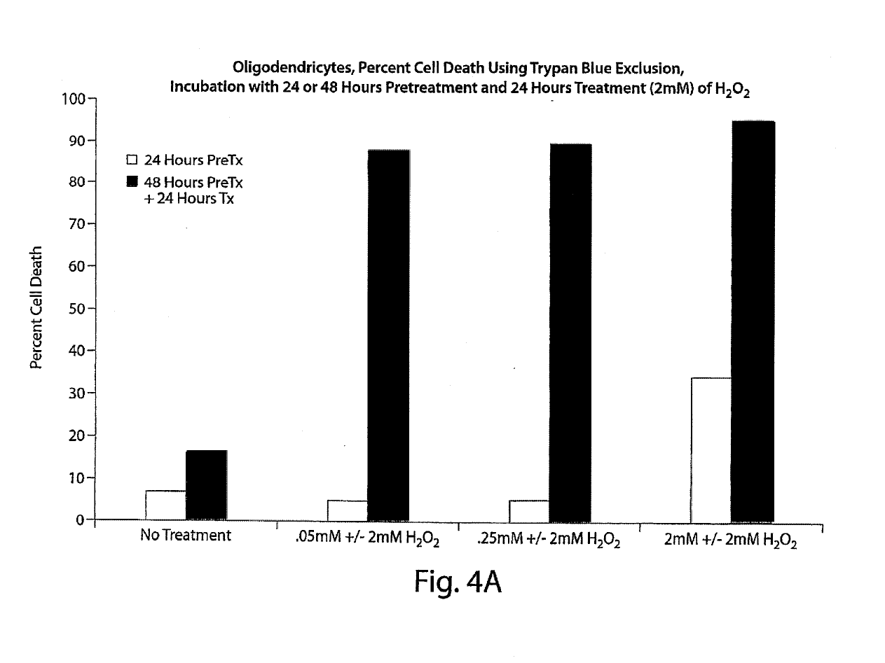

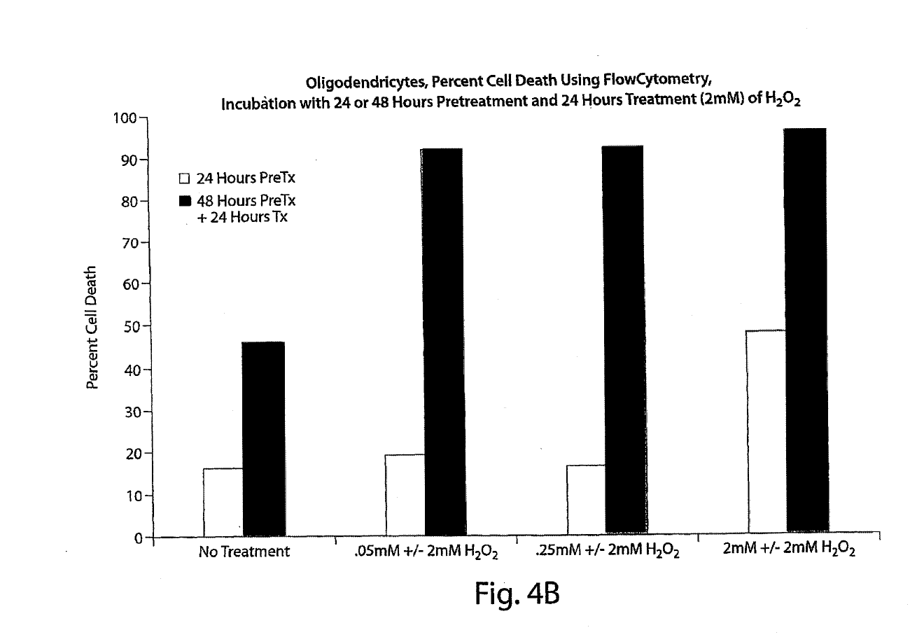

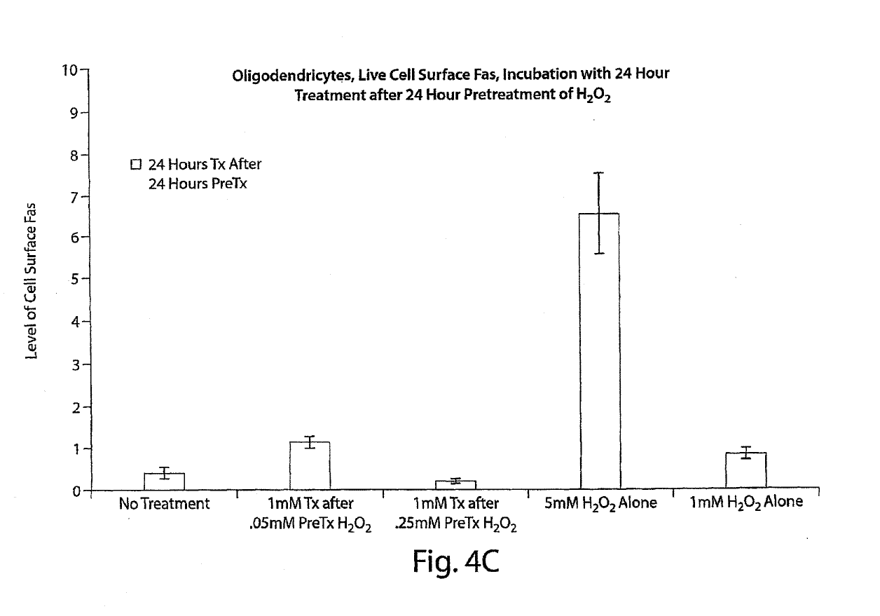

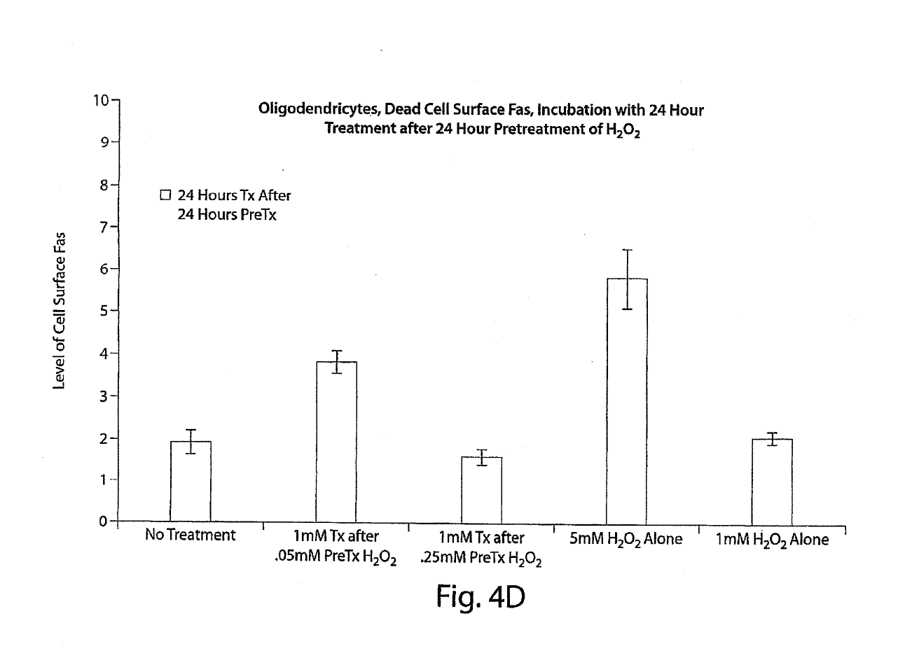

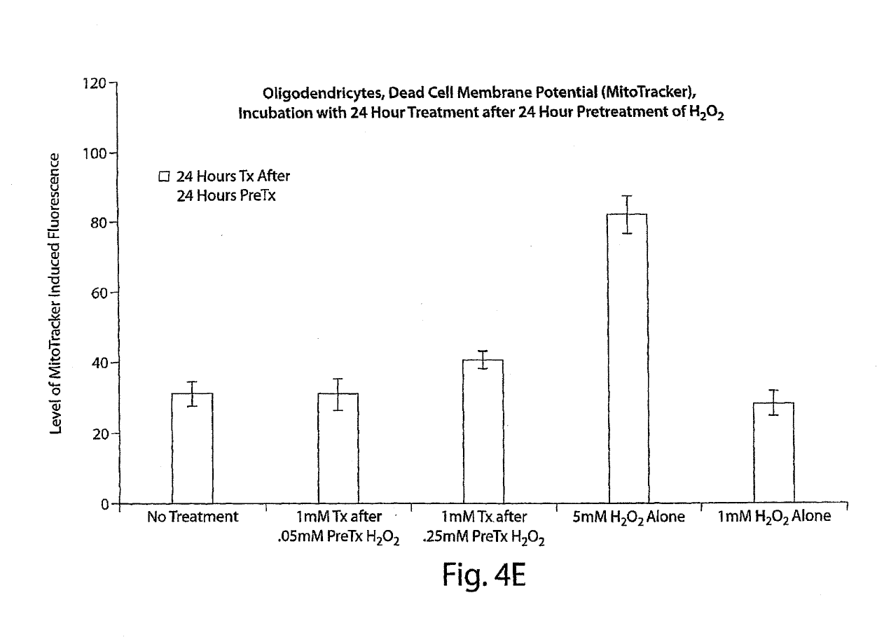

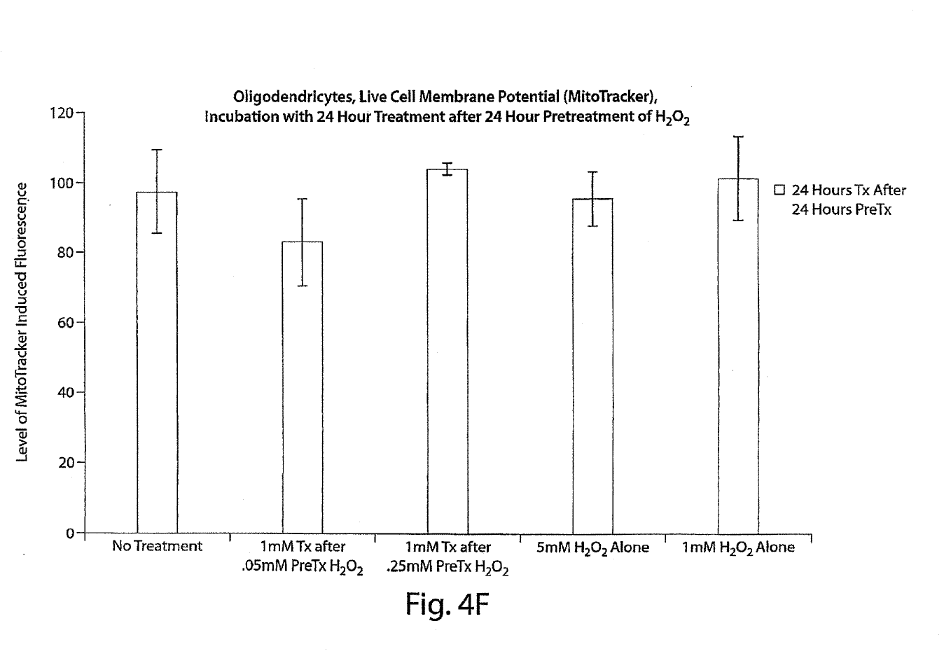

Mouse oligodendrocyte cells (a kind gift from Dr. Adrian Cameron, University of Kentucky) were cultured as described in the Methods. The cells were pre-treated or not with H₂O₂ at the concentration indicated on the graphs in FIG. 4. Following pre-treatment the cells were incubated with higher concentrations of H₂O₂ for an additional time frame (indicated on the graphs, FIG. 4a and FIG. 4b). The measurements were assessed using Trypan Blue exclusion (FIG. 4a and FIG. 4b) and Mitotracker (FIG. 4e and FIG. 4f). The cells were harvested and stained with Anti-Fas (CD95) antibody (Pharmingen) as indicated. Expression of Fas was measured on both live (FIG. 4c) and dead cell (FIG. 4d) populations.

### Example 10

### Assessment of Cell Death, Cell Surface Fas, and Mitotracker Fluorescence in Rat Pheochromocytoma Cells in Response to H₂O₂

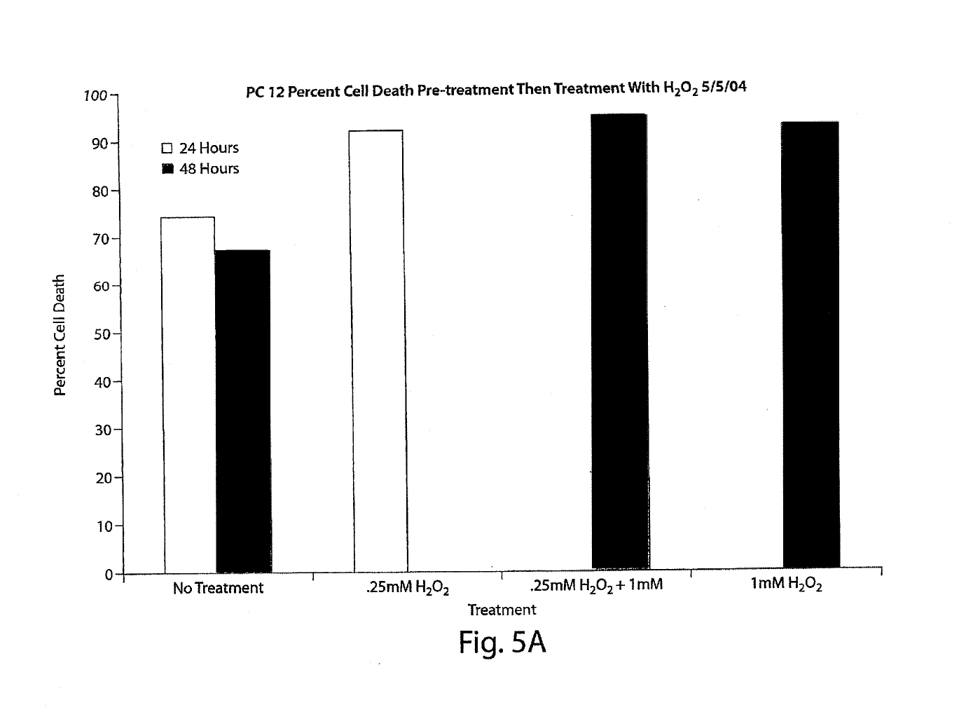

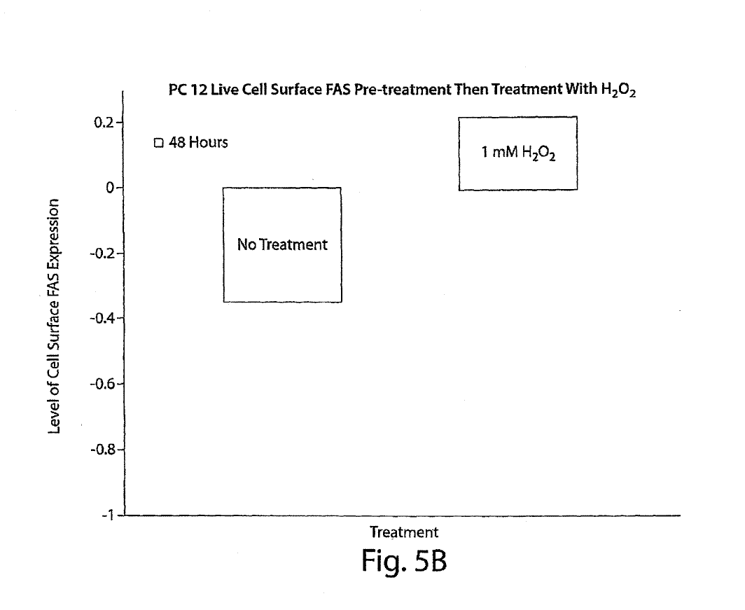

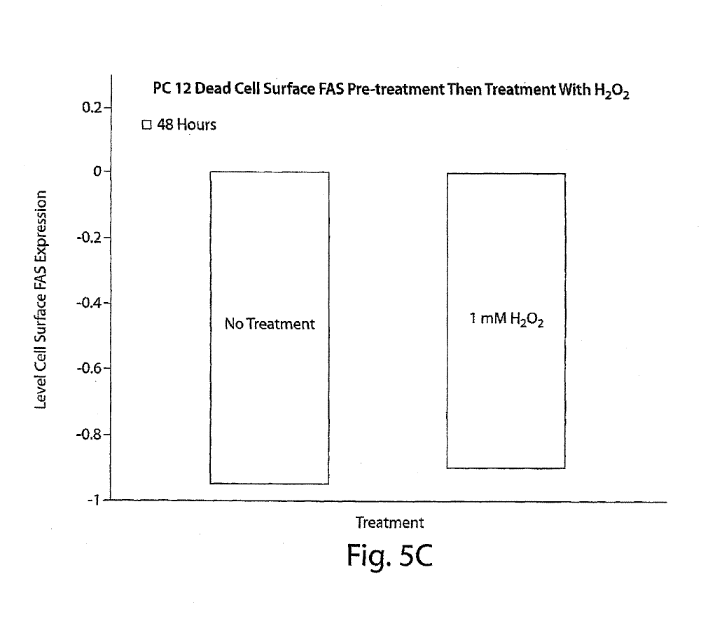

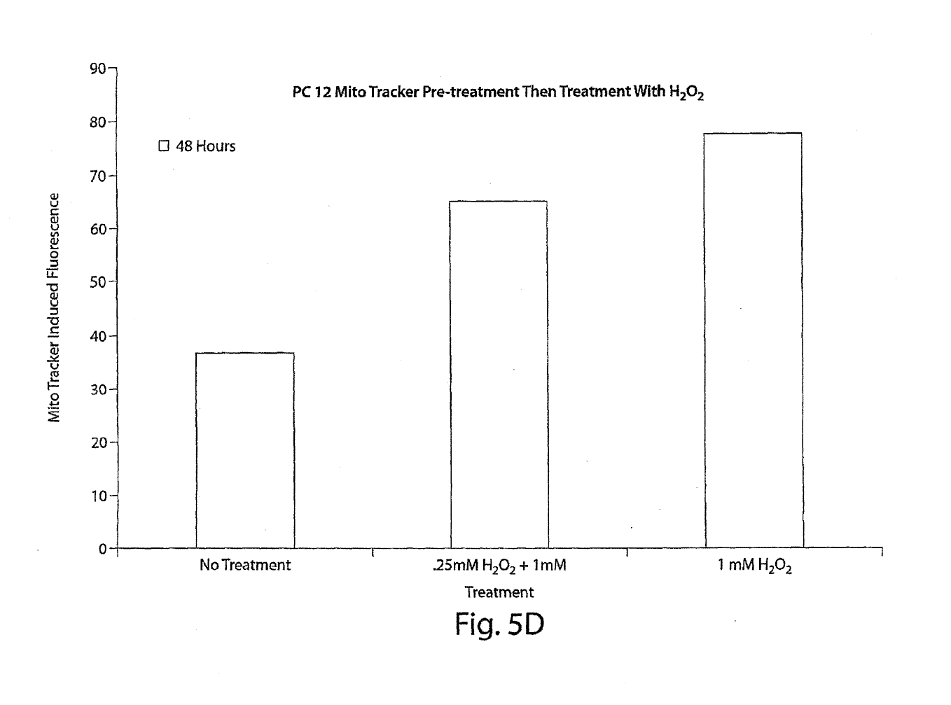

Rat pheochromocytoma cells (ATCC) were cultured as described in, the Methods. The cells were pre-treated or not with H₂O₂ at the concentration indicated on the graphs, FIG. 5. Following pre-treatment the cells were incubated with a higher concentration of H₂O₂ for an additional time frame (indicated on the graphs, FIG. 5). The cells were harvested and stained with Anti-Fas (CD95) antibody (Pharmingen) as indicated (FIGS. 5b and 5C). Expression of Fas was measured on both live and dead cell populations. They were also analyzed for percent death flow cytometrically (FIG. 5a) as described in the Methods. The cells were also stained with the fluorescent probe MitoTracker Red (Molecular Probes, Eugene, Oreg., FIG. 5d) as described in the Methods.

All references, patents and patent publications that are recited in this application, including U.S. Provisional Patent Application No. 60/470,046 to which the instant application claims priority, are incorporated in their entirety herein by reference.

We claim:

1-15. (canceled)

16. A modified stem cell, comprising a stem cell having a heterologous mitochondria.

17. The modified stem cell of claim 16 wherein the heterologous mitochondria has a level of mitochondrial membrane potential that is within a normal range relative to a healthy stem cell.

18. A method for promoting tissue generation, comprising subjecting the modified stem cell of claim 14 to growth promoting conditions.

19. The method of claim 18, wherein the modified stem cell is implanted into a subject.

20. The method of claim 19 wherein the modified stem cell is autologous to the subject.

21. The method of claim 18, wherein the stem cell is a neural stem cell.

22-36. (canceled)

36. A method of generating neural tissue comprising implanting a neural stem cell of claim 32 into a subject, inducing expression of the UCP2 gene to grow neural tissue, and inducing expression of the UCP4 gene to differentiate the neural stem cells into neural tissue.

37. A modified oocyte or embryonic cell, comprising:
an oocyte or embryonic cell having a microinjected heterologous mitochondria.

38. (canceled)
